(19) United States
(12) Patent Application Publication
(10) Pub. No.: US 2007/0225340 A1
(43) Pub. Date: Sep. 27, 2007

(54) Phenyl Compounds And Their Use In The Treatment Of Conditions Mediated By The Action Of Pge2 At The Ep1 Receptor

(75) Inventors: Rino Antonio Bit, Essex (GB); Gerard Martin Paul Giblin, Essex (GB); Adrian Hall, Essex (GB); Thomas Hayhow, Essex (GB); David Nigel Hurst, Essex (GB); Ian Reginald Kilford, Essex (GB); Neil Derek Miller, Essex (GB); Alan Naylor, Essex (GB); Riccardo Novelli, Essex (GB); Tiziana Scoccitti, Essex (GB)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(73) Assignee: Glaxo Group Limited, Greenford, Middlesex (GB)

(21) Appl. No.: 11/568,573

(22) PCT Filed: Apr. 29, 2005
(86) PCT No.: PCT/EP05/04726

§ 371 Date: Nov. 2, 2006

(30) Foreign Application Priority Data
May 6, 2004 (GB) 0410121.8

(51) Int. Cl. A61K 31/4418; C07D 213/55

(52) U.S. Cl. 514/354; 546/342

(57) ABSTRACT

Compounds of formula (I) or a pharmaceutically acceptable derivative thereof: 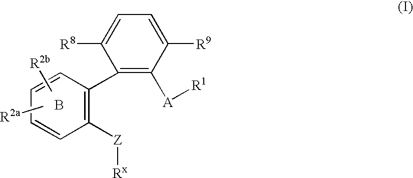
wherein A, B, Z, R¹, R^{2a}, R^{2b}, R^{x}, R⁸, and R⁹ are as defined in the specification, a process for the preparation of such compounds, pharmaceutical compositions comprising such compounds and the use of such compounds in medicine.

This invention relates to phenyl derivatives, to processes for their preparation, to pharmaceutical compositions containing them and to their use in medicine, in particular their use in the treatment of conditions mediated by the action of PGE₂ at the EP₁ receptor.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion. We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S Narumiya et al, Physiological Reviews 1999, 79(4), 1193-126. An article from The British Journal of Pharmacology (1994, 112, 735-740) suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation (2001, 107 (3), 325) shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, inhibit hormone-induced uterine contractions and have anti-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

In The American Physiological Society (1994, 267, R289-R-294), studies suggest that PGE₂-induced hyperthermia in the rat is mediated predominantly through the EP₁ receptor.

WO 96/06822 (Mar. 7, 1996), WO 96/11902 (Apr. 25, 1996), EP 752421-A1 (Jan. 08, 1997), WO 01/19814 (22 Mar. 2001), WO 03/084917 (16 Oct. 2003), WO 03/101959 (11 Dec. 2003), WO 2004/039753 (13 May 2004) and WO 2004/083185 (30 Sep. 2004) disclose compounds as being useful in the treatment of prostaglandin mediated diseases.

P. Lacombe et al (220th National Meeting of The American Chemical Society, Washington D.C., USA, 20-24 Aug., 2000) disclosed 2,3-diarylthiophenes as ligands for the human EP₁ prostanoid receptor. Y. Ducharme et al (18^{th} International Symposium on Medicinal Chemistry; Copenhagen, Denmark and Malmo, Sweden; 15^{th}-19^{th} Aug. 2004) disclosed 2,3-diarylthiophenes as EP₁ receptor antagonists. Y. Ducharme et al, Biorg. Med. Chem. Lett., 2005, 15(4): 1155 also discloses 2,3-diarylthiophenes as selective EP₁ receptor antagonists.

It is now suggested that a novel group of phenyl derivatives surprisingly are selective for the EP₁ receptor over the EP₃ receptor, and are therefore indicated to be useful in treating conditions mediated by the action of PGE₂ at EP₁ receptors. Such conditions include pain, or inflammatory, immunological, bone, neurodegenerative or renal disorders.

Accordingly the present invention provides a compound of formula (I): 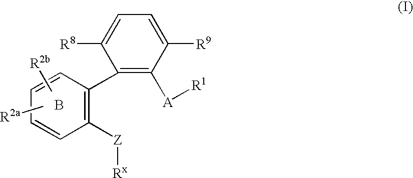
wherein:
A is an optionally substituted aryl, or an optionally substituted 5- or 6-membered heterocyclyl ring, or an optionally substituted bicyclic heterocyclyl group;
B is a phenyl or pyridyl ring;
Z is O, S, SO, or SO₂;
R¹ is CO₂H, CN, CONR⁵R⁶, CH₂CO₂R⁴, SO₂NR⁵R⁶, NR⁴CONR⁵R⁶, COalkyl, 2H-tetrazol-5-yl-methyl, optionally substituted SO₂alkyl, optionally substituted bicyclic heterocycle or optionally substituted heterocyclyl;
R^{2a} and R^{2b} are each independently selected from hydrogen, halogen, CONR⁵R⁶, CN, SO₂alkyl, SR⁵, NO₂, optionally substituted alkyl, optionally substituted alkoxy, optionally substituted aryl, and optionally substituted heteroaryl;
R^{x} is optionally substituted alkyl wherein 1 or 2 of the non-terminal carbon atoms are optionally replaced by a group independently selected from NR⁴, O and SOₙ, wherein n is 0, 1 or 2; optionally substituted alkenyl; or optionally substituted alkynyl: or R^{x} is optionally substituted CQ^{a}Q^{b}-heterocyclyl; optionally substituted CQ^{a}Q^{b}-bicyclic heterocyclyl; or optionally substituted CQ^{a}Q^{b}-aryl;
R⁴ is hydrogen or an optionally substituted alkyl;
R⁵ is hydrogen or an optionally substituted alkyl;
R⁶ is hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ is optionally substituted alkyl, optionally substituted heteroaryl or optionally substituted aryl;
R⁸ and R⁹ are each independently selected from hydrogen, halogen, CF₃, C₁₋₃alkoxy and C₁₋₃alkyl;
Q^{a} and Q^{b} are each independently selected from hydrogen and CH₃;
   and derivatives thereof;
   wherein:
when A is a 6-membered ring the R¹ substituent and the above-defined 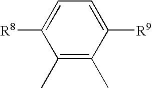
group are attached to carbon atoms 1,2-, or 1,3- relative to each other, and when A is a five-membered ring or bicyclic heterocyclyl group the R¹ substituent and the above-defined 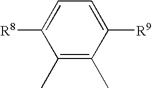
group are attached to substitutable carbon atoms 1,2- or 1,3- relative to each other; and
neither R^{2a} nor R^{2b} are ortho to the Z substituent; and
R^{x} is not 4-(tert-butyl)benzyl or 4-methylpent-1-yl; and
when R¹ is CONH₂, it is ortho to the 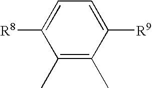
group; and
when R¹ is SO₂NR⁵R⁶, then R⁵ is hydrogen and R⁶ is COR⁷;
   provided that the compound of formula (I) is not:
2-benzyloxy-5-chloro-[1,1′;2′,1″]terphenyl-3″-carboxylic acid;
(2-benzyloxy-5-chloro-[1,1′;2′,1″]terphenyl-3″-yl)-acetic acid;
(2-benzyloxy-5-chloro[1,1′;2′,1″]terphenyl-2″-yl)acetic acid;
(2-benzyloxy-5-chloro[1,1′;2′,1″]terphenyl-4″-yl)acetic acid;
5″-acetylamino-2-benzyloxy-5-chloro[1,1′;2′,1″]terphenyl-3″-carboxylic acid;
2-benzyloxy-5-chloro-5″-propionylamino[1,1′;2′,1″]terphenyl-3″-carboxylic acid;
2-benzyloxy-5-chloro-5″-(2-methylpropanoylamino)-[1,1′;2′,1″]terphenyl-3″-carboxylic acid;
2-benzoyloxy-5″-butyrylamino-5-chloro[1,1′;2′,1″]terphenyl-3″-carboxylic acid;
2-benzyloxy-5-chloro-5″-[(1-phenyl-methanoyl)amino]-[1,1′;2′,1″]terphenyl-3″-carboxylic acid;
2-benzyloxy-5-chloro-5″-methanesulfonylamino-[1,1′;2′,1″]terphenyl-3″-carboxylic acid 5″-amino-2-benzyloxy-5-chloro[1,1′;2′,2″]-3″-carboxylic acid;
2-benzyloxy-5″-butyrylamino-5-trifluoromethyl[1,1′;2′,1″]terphenyl-3″-carboxylic acid-3-carboxylic acid;
2-benzyloxy-4″-chloro[1,1′;2′,1″]terphenyl 2″-carboxylic acid;
2-benzyloxy-5″-fluoro-[1,1′;2′,1″]terphenyl-2″-carboxylic acid;
2-benzyloxy-4″-fluoro-[1,1′;2′,1″]terphenyl-2″-carboxylic acid;
2″-benzyloxy-5-fluoro-[1,1′;2′,1″]terphenyl-3-carboxylic acid;
4″-amino-2-benzyloxy-[1,1′;2′,1″]terphenyl-3″-carboxylic acid;
5″-acetylamino-2-benzyloxy-[1,1′;2′,1″]terphenyl-2″-carboxylic acid;
2-benzyloxy-5-chloro-[1,1′;2′,1″]terphenyl-2″-carboxylic acid;
2-benzyloxy-[1,1′;2′,1″]terphenyl-3″-carboxylic acid;
2-benzyloxy-5-chloro-[1,1′;2′,1″]terphenyl-2″-carboxylic acid amide;
5-(2-benzyloxy-5-chloro-[1,1′;2′,1″]terphenyl-3″-yl)-1H-tetrazole;
N-[1-(2-benzyloxy-5-chloro-[1,1′;2′,1″]terphenyl-2″-yl)-methanoyl]-benzenesulfonamide;
2-benzyloxy-[1,1′;2′,1″]terphenyl-4″-sulfonic acid (1-phenyl-methanoyl)-amide;
2-benzyloxy-[1,1′;2′,1″]terphenyl-4″-sulfonic acid [1-(4-nitro-phenyl)-methanoyl]-amide;
2-benzyloxy-[1,1′;2′,1″]terphenyl-3″-sulfonic acid acetyl-amide;
5-chloro-2-(3-methyl-butoxy)-[1,1′;2′,1″]terphenyl-3″-carboxylic acid;
5-chloro-2-(4-fluoro-benzyloxy)-[1,1′;2′,1″]terphenyl-3″-carboxylic acid;
5-chloro-2-(2,4-difluoro-benzyloxy)-[1,1′;2″,1″]terphenyl-3″-carboxylic acid;
5-chloro-2-(4-chloro-benzyloxy)-[1,1′;2′,1″]terphenyl-3″carboxylic acid;
5-chloro-2-(2-fluoro-4-chloro-benzyloxy)-[1,1′;2′,1″]terphenyl-3″-carboxylic;
5-chloro-2-(4-isobutoxy)-[1,1′,2′,1″]terphenyl-3″-carboxylic acid;
5-chloro-2-(pyridin-2-ylmethoxy)-[1,1′;2′,1″]terphenyl-3″-carboxylic acid;
5-chloro-2-(pyridin-4-ylmethoxy)-[1,1′;2′,1″]terphenyl-3″-carboxylic acid;
5-chloro-2-(pyridin-3-ylmethoxy)-[1,1′;2′,1″]terphenyl-3″-carboxylic acid;
5-chloro-2-cyclohexylmethoxy-[1,1′;2′,1″]terphenyl-3′-′carboxylic acid;
5-chloro-2-(thiophen-3-ylmethoxy)-[1,1′;2′,1″]terphenyl-3″-carboxylic acid;
5-chloro-2-(thiophen-2-ylmethoxy)-[1,1′;2′,1″]terphenyl-3″-carboxylic acid;
5-chloro-2-cyclopentylmethoxy-[1,1′;2′,1″]terphenyl-3″-carboxylic acid;
5-chloro-2-propoxy-[1,1′;2′,1″]terphenyl-3″-carboxylic acid;
2-butoxy-5-chloro-[1,1′;2′,1″]terphenyl-3″-carboxylic acid;
5-chloro-2-isopropoxy-[1,1′;2′,1″]terphenyl-3″-carboxylic acid;
5-chloro-2-isobutoxy-[1,1′;2′,1″]terphenyl-2″-carboxylic acid;
2″-{[(4-fluorophenyl)methyl]oxy}-5-[(methyloxy)carbonyl]-5″-(trifluoromethyl)-1,1′:2′,1″-terphenyl-3-carboxylic acid;
5-chloro-2″-[(phenylmethyl)oxy]-1,1′:2′,1″-terphenyl-2-carboxylic acid;
4-(methoxy)-2″-[(phenylmethyl)oxy]-1,1′:2′,1″-terphenyl-2-carboxylic acid;
2″-{[(2,4-difluorophenyl)methyl]oxy}-4-(propanoylamino)-5″-(trifluoromethyl)-1,1′:2′,1″-terphenyl-2-carboxylic acid;
2″-{[(2,4-difluorophenyl)methyl]oxy}-4-[(2-methylpropanoyl)amino]-5″-(trifluoromethyl)-1,1′:2′,1″-terphenyl-2-carboxylic acid;
5-(2-oxo-1-pyrrolidinyl)-2″-[(phenylmethyl)oxy]-5″-(trifluoromethyl)-1,1′:2′,1″-terphenyl-3-carboxylic acid;
2″-{[(4-fluorophenyl)methyl]oxy}-5″-(trifluoromethyl)-1,1′:2′,1″-terphenyl-3,5-dicarboxylic acid;
2″-{[(4-fluorophenyl)methyl]oxy}-5-{[(2-methylpropyl)amino]carbonyl}-5″-(trifluoromethyl)-1,1′:2′,1″-terphenyl-3-carboxylic acid;
6-[2′-{[(4-fluorophenyl)methyl]oxy}-5′-(trifluoromethyl)-2-biphenylyl]-2-pyrazinecarboxylic acid;
2″-{[(4-fluorophenyl)methyl]oxy}-5-(propanoylamino)-5″-(trifluoromethyl)-1,1′:2′,1″-terphenyl-3-carboxylic acid;
2″-[(phenylmethyl)oxy]-5-(propanoylamino)-5″-(trifluoromethyl)-1,1′:2′,1″-terphenyl-3-carboxylic acid;
2″-{[(2,4-difluorophenyl)methyl]oxy}-5-(propanoylamino)-5″-(trifluoromethyl)-1,1′:2′,1″terphenyl-3-carboxylic acid;
5″-chloro-5-{[(methyloxy)acetyl]amino}-2″-[(phenylmethyl)oxy]-1,1′:2′,1″-terphenyl-3-carboxylic acid;
5″-chloro-2″-[(phenylmethyl)oxy]-5-[(2-thienylacetyl)amino]-1,1′:2′,1″-terphenyl-3-carboxylic acid;
5″-chloro-2″-[(phenylmethyl)oxy]-5-({[(phenylmethyl)oxy]acetyl}amino)-1,1′:2′,1″-terphenyl-3-carboxylic acid;
5-{[(1-acetyl-4-piperidinyl)carbonyl]amino}-5″-chloro-2″-[(phenylmethyl)oxy]-1,1′:2′,1″-terphenyl-3-carboxylic acid;
5″-chloro-5-[(phenylacetyl)amino]-2″-[(phenylmethyl)oxy]-1,1′:2′,1″-terphenyl-3-carboxylic acid;
5″-chloro-5-{[(3,5-dimethyl-4-isoxazolyl)carbonyl]amino}-2″-[(phenylmethyl)oxy]-1,1′:2′,1″-terphenyl-3-carboxylic acid;
5″-chloro-5-[(3-methylbutanoyl)amino]-2″-[(phenylmethyl)oxy]-1,1′:2′,1″-terphenyl-3-carboxylic acid;
5″-chloro-5-(glycylamino)-2″-[(phenylmethyl)oxy]-1,1′:2′,1″-terphenyl-3-carboxylic acid;
2″-[(penylmethyl)oxy]-4-(propanoylamino)-1,1′:2′,1″-terphenyl-2-carboxylic acid;
4-[(2-methylpropanoyl)amino]-2″-[(phenylmethyl)oxy]-1,1′:2′,1″-terphenyl-2-carboxylic acid;
5-cyano-2″-{[(2,4-difluorophenyl)methyl]oxy}-5″-(trifluoromethyl)-1,1′:2′,1″-terphenyl-3-carboxylic acid;
5″-bromo-5-cyano-2″-[(phenylmethyl)oxy]-1,1′:2′,1″-terphenyl-3-carboxylic acid;
5-cyano-2″-[(phenylmethyl)oxy]-5″-(trifluoromethyl)-1,1′:2′,1″-terphenyl-3-carboxylic acid;
5-(aminocarbonyl)-2″-{[(4-fluorophenyl)methyl]oxy}-5″-(trifluoromethyl)-1,1′:2′,1″-terphenyl-3-carboxylic acid;
2″-{[(4-fluorophenyl)methyl]oxy}-5-{[(2-hydroxyethyl)amino]carbonyl}-5″-(trifluoromethyl)-1,1′:2′,1″-terphenyl-3-carboxylic acid;
2″-{[(4-fluorophenyl)methyl]oxy}-5-{[(3-pyridinylmethyl)amino]carbonyl}-5″-(trifluoromethyl)-1,1′:2′,1″-terphenyl-3-carboxylic acid;
6-{5′-chloro-2′-[(phenylmethyl)oxy]-2-biphenylyl}-2-pyridinecarboxylic acid;
6-(5′-chloro-2′-{[(4-fluorophenyl)methyl]oxy}-2-biphenylyl)-2-pyridinecarboxylic acid;
6-(5′-chloro-2′-{[(2,4-difluorophenyl)methyl]oxy}-2-biphenylyl)-2-pyridinecarboxylic acid;
2-[2′-{[(4-fluorophenyl)methyl]oxy}-5′-(trifluoromethyl)-2-biphenylyl]-4-pyridinecarboxylic acid;
3-amino-6-[2′-{[(4-fluorophenyl)methyl]oxy}-5′-(trifluoromethyl)-2-biphenylyl]-2-pyrazinecarboxylic acid;
4-(acetylamino)-2″-[(phenylmethyl)oxy]-5″-(trifluoromethyl)-1,1′:2′,1″-terphenyl-2-carboxylic acid;
4-(acetylamino)-2″-{[(4-fluorophenyl)methyl]oxy}-5″-(trifluoromethyl)-1,1′:2′,1″-terphenyl-2-carboxylic acid;
4-(acetylamino)-2″-{[(2,4-difluorophenyl)methyl]oxy}-5″-(trifluoromethyl)-1,1′:2′,1″-terphenyl-2-carboxylic acid;
4-methyl-2″-[(phenylmethyl)oxy]-1,1′:2′,1″-terphenyl-2-carboxylic acid;
2″-{[(2,4-difluorophenyl)methyl]oxy}-4-methyl-5″-(trifluoromethyl)-1,1′:2′,1″-terphenyl-2-carboxylic acid;
2-benzyloxy-5-fluoro[1,1′,2′,2″]terphenyl-3″-carboxylic acid;
2-(4-fluorobenzyl)oxy-5-fluoro[1,1′,2,2′]terphenyl-3″-carboxylic acid;
2-(2,4-difluorobenzyl)oxy-5-fluoro[1,1′,2,2′]terphenyl-3″-carboxylic acid;
2′-{5-chloro-2-(benzyloxy)-3-pyridinyl]-3-biphenylcarboxylic acid;
2′-{6-chloro-3-[(phenylmethyl)oxy]-2-pyridinyl}-3-biphenylcarboxylic acid;
5-amino-2′-{6-chloro-3-[(phenylmethyl)oxy]-2-pyridinyl}-3-biphenylcarboxylic acid;
5-{2′-[(phenylmethyl)oxy]-2-biphenylyl}-3-pyridinecarboxylic acid;
4″-chloro-2″-[(phenylmethyl)oxy]-1,1′:2′,1″-terphenyl-2-carboxylic acid; or
6″-fluoro-2-benzyloxy-[1,1′;2′,1″]terphenyl-3″-carboxylic acid.

Suitably A is a six-membered ring. More suitably A is optionally substituted phenyl or optionally substituted pyridyl. In one aspect A is pyridyl.

Suitably B is phenyl.

Optional substituents for A may include up to four substituents, preferably 0 or 1 substituent, independently selected from halogen, NH₂, NHC₁₋₆alkyl, OH, optionally substituted OC₁₋₆alkyl, and optionally substituted C₁₋₆alkyl.

In one aspect A does not have additional substituents.

Suitably Z is O.

Suitably R¹ is CO₂H.

Suitably R^{2a} is hydrogen.

Suitably R^{2b} represents hydrogen, halogen e.g. fluorine, bromine or chlorine, or CF₃. Preferably R^{2b} represents bromine or chlorine. In one aspect R^{2b} represents bromine. In another aspect R^{2b} represents chlorine.

Preferably R^{2b} is positioned 1,4- relative to the Z substituent and 1,3- relative to the phenyl ring.

Suitably R^{x} represents optionally substituted C₂₋₈alkyl; optionally substituted C₂₋₈alkenyl; or optionally substituted C₂₋₈alkynyl: or R^{x} represents optionally substituted CQ^{a}Q^{b}-heterocyclyl, optionally substituted CQ^{a}Q^{b}-bicyclic heterocyclyl, or optionally substituted CQ^{a}Q^{b}-aryl, wherein Q^{a} and Q^{b} are independently selected from hydrogen and CH₃.

Suitably Q^{a} is hydrogen.

Suitably Q^{b} is hydrogen.

When R^{x} is optionally substituted CQ^{a}Q^{b}-aryl, suitably it is optionally substituted CH₂-phenyl.

In one aspect R^{x} is optionally substituted C₂₋₈alkyl, e.g. optionally substituted C₃₋₈alkyl; optionally substituted C₂₋₈alkenyl, e.g optionally substituted C₃₋₈alkenyl or optionally substituted C₂₋₈alkynyl, e.g or optionally substituted C₃₋₈alkynyl: or R^{x} represents optionally substituted CH₂-heterocyclyl or optionally substituted CH₂-phenyl.

In another aspect R^{x} is optionally substituted C₁₋₈alkyl, e.g. optionally substituted C₃₋₈alkyl; optionally substituted C₂₋₈alkenyl, e.g optionally substituted C₃₋₈alkenyl or optionally substituted C₂₋₈alkynyl, e.g or optionally substituted C₃₋₈alkynyl: or R^{x} represents optionally substituted CH₂-heterocyclyl.

In yet another aspect R^{x} is optionally substituted C₁₋₈alkyl

Suitable values when R^{x} is C₁₋₈alkyl include propyl, 1-methylethyl, 2-methylpropyl, 2-methylbutyl, 3-methylbutyl, 2,2-dimethylpropyl, 2-ethylbutyl, cyclopropylmethyl, cyclobutylmethyl, cyclopentylmethyl and cyclohexylmethyl. Other suitable values include methylcyclopropylmethyl, butyl, and cyclopentyl,

When R^{x} is alkyl, suitable optional substituents include fluoro. Substituted alkyl includes, for example, 2,2,2-trifluoroethyl and 3,3,3-trifluoropropyl. Other substituted alkyl groups include trifluoromethylcyclopropylmethyl.

Suitable values when R^{x} is C₂₋₈alkenyl include 2-methyl-2-propen-1-yl. Other suitable values include 2-propen-1-yl, 2-methyl-2-buten-1-yl, 2-buten-1-yl, and cyclopentenylmethyl.

When R^{x} is alkenyl, suitable optional substituents include fluoro and chloro. Substituted alkenyl includes, for example, 2-chloro-2-propen-1-yl.

Suitable values when R^{x} is optionally substituted alkynyl include optionally substituted 2-propyn-1-yl, e.g. 2-propyn-1-yl and 3-phenyl-2-propyn-1-yl; 2-butyn-1-yl; and 2-pentyn-1-yl.

Suitable values when R^{x} is CQ^{a}Q^{b}heterocyclyl include CH₂tetrahydrofuranyl and CH₂tetrahydropyranyl, e.g. tetrahydro-2-furanylmethyl, tetrahydro-3-furanylmethyl and tetrahydro-2H-pyran-4-ylmethyl.

Suitably optional substituents for R^{x} when CQ^{a}Q^{b}phenyl include one to four substituents selected from Cl, F, Br, and CF₃. Particular examples include Cl, F and CF₃.

Suitably R⁴ includes hydrogen and C₁₋₆alkyl. More suitably R⁴ is hydrogen.

Suitably R⁵ includes hydrogen and C₁₋₆alkyl. More suitably R⁵ is selected from hydrogen and C₁₋₃alkyl.

Suitably R⁶ is hydrogen.

Suitably R⁷ is C₁₋₆alkyl.

Suitably R⁸ and R⁹ are each independently selected from hydrogen, Cl, F, CF₃, OCH₃ and CH₃. In one alternative R⁸ and R⁹ each represent hydrogen.

In one aspect R⁸ is hydrogen and R⁹ is C₁₋₃alkyl, e.g. CH₃.

In a further aspect the present invention provides a compound of formula (I) which is a compound of formula (Ia): 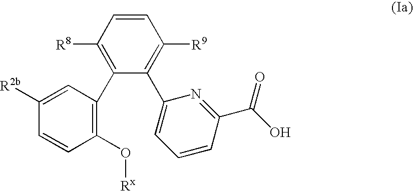
wherein:
R^{2b} is selected from CF₃, chloro and bromo;
R^{x} is optionally substituted alkyl wherein 1 or 2 of the non-terminal carbon atoms are optionally replaced by a group independently selected from NR⁴, O and SOₙ, wherein n is 0, 1 or 2; optionally substituted alkenyl; or optionally substituted alkynyl: or R^{x} is optionally substituted CQ^{a}Q^{b}-heterocyclyl; optionally substituted CQ^{a}Q^{b}-bicyclic heterocyclyl; or optionally substituted CQ^{a}Q^{b}-aryl;
R⁴ is hydrogen or C₁₋₆alkyl;
R⁸ and R⁹ are independently selected from hydrogen, halogen, CF₃, C₁₋₃alkoxy and C₁₋₃alkyl;
Q^{a} and Q^{b} are independently selected from hydrogen and CH₃;
   and derivatives thereof;
   with the proviso that:
R^{x} is not 4-(tert-butyl)benzyl or 4-methylpent-1-yl; and
   the compound of formula (la) is not:
6-{5′-chloro-2′-[(phenylmethyl)oxy]-2-biphenylyl}-2-pyridinecarboxylic acid;
6-(5′-chloro-2′-{[(4-fluorophenyl)methyl]oxy}-2-biphenylyl)-2-pyridinecarboxylic acid; or
6-(5′-chloro-2′-{[(2,4-difluorophenyl)methyl]oxy}-2-biphenylyl)-2-pyridinecarboxylic acid.

In one aspect R^{2b} is CF₃ or bromine. In a particular aspect R^{2b} is chlorine. In an alternative aspect R^{2b} is bromine.

Suitably R⁸ and R⁹ each represent hydrogen.

Suitably R^{x} includes optionally substituted C₂₋₈alkyl, optionally substituted C₂₋₈alkenyl, or optionally substituted C₂₋₈alkynyl; or R^{x} represents optionally substituted CH₂-heterocyclyl or optionally substituted CH₂-phenyl.

In one aspect R^{x} includes optionally substituted C₃₋₈alkyl, optionally substituted C₃₋₈alkenyl, or optionally substituted C₃₋₈alkynyl: or optionally substituted CH₂-heterocyclyl.

In a further aspect R^{x} includes optionally substituted C₃₋₈alkyl.

In another aspect R^{x} represents CH₂-phenyl optionally substituted by one, two, or three substituents independently selected from CF₃, Br, Cl and F.

In a yet further aspect R^{2b} is chlorine and R^{x} is optionally substituted C₃₋₈alkyl, optionally substituted C₃₋₈alkenyl, or optionally substituted C₃₋₈alkynyl, or optionally substituted CH₂-heterocyclyl, especially optionally substituted C₃₋₈alkyl.

In another aspect the present invention provides a compound of formula (I) which is a compound of formula (Ib): 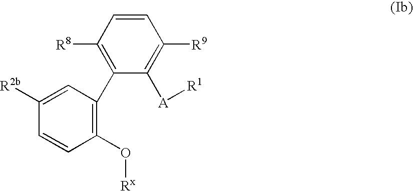
wherein:
A represents an optionally substituted aryl, or an optionally substituted 5- or 6-membered heterocyclyl ring, or an optionally substituted bicyclic heterocyclyl group;
R¹ is CO₂H;
R^{2b} is selected from CF₃, chloro and bromo;
R^{x} represents optionally substituted alkyl wherein 1 or 2 of the non-terminal carbon atoms may also be optionally replaced by a group independently selected from NR⁴, O and SOₙ, wherein n is 0, 1 or 2: or R^{x} represents optionally substituted CQ^{a}Q^{b}-heterocyclyl, optionally substituted CQ^{a}Q^{b}-bicyclic heterocyclyl, optionally substituted CQ^{a}Q^{b} alkenyl, optionally substituted CQ^{a}Q^{b}alkynyl, or optionally substituted CQ^{a}Q^{b}-aryl;
R⁴ is hydrogen or C₁₋₆alkyl;
R⁸ represents hydrogen, halogen, CF₃, C₁₋₃alkoxy or C₁₋₃alkyl;
R⁹ represents halogen, CF₃, C₁₋₃alkoxy or C₁₋₃alkyl; and
Q^{a} and Q^{b} are independently selected from hydrogen and CH₃;
   and derivatives thereof;
   with the proviso that R^{x} is not 4-(tert-butyl)benzyl or 4-methylpent-1-yl.

Suitably A is the moiety 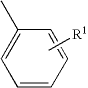
wherein R¹ is CO₂H.

In one aspect R^{2b} is chloro.

In another aspect R^{2b} is selected from CF₃ and bromo, especially bromo.

Suitably R⁸ is hydrogen and R⁹ is C₁₋₃alkyl or halogen. In a particular aspect R⁸ is hydrogen and R⁹ is CH₃.

Suitably R^{x} is benzyl.

Examples of the compounds of formula (I) include the compounds of Examples 1 to 70 and derivatives thereof.

Preferred examples include 6-{5′-chloro-2′-[(2-methylpropyl)oxy]-2-biphenylyl}-2-pyridinecarboxylate and derivatives thereof. A particular derivative is the sodium salt.

The compounds of the invention are selective for EP₁ over EP₃. Certain compounds of the Examples are 100 fold selective for EP₁ over EP₃.

Derivatives of the compounds of formula (I) include pharmaceutically acceptable derivatives.

The invention is described using the following definitions unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I).

It will be appreciated by those skilled in the art that the compounds of formula (I) may be modified to provide pharmaceutically acceptable derivatives thereof at any of the functional groups in the compounds, and that the compounds of formula (I) may be derivatised at more than one position.

It will be appreciated that, for pharmaceutical use, the salts referred to above will be pharmaceutically acceptable salts, but other salts may find use, for example in the preparation of compounds of formula (I) and the pharmaceutically acceptable salts thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. A particular salt is the sodium salt. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropyl amine, tris(hydroxymethyl)aminomethane, and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The terms “halogen or halo” are used to represent fluorine, chlorine, bromine or iodine, more preferably fluorine, chlorine and bromine.

The term “alkyl” as a group or part of a group means a straight, branched or cyclic chain alkyl group or combinations thereof. Unless hereinbefore defined, examples of alkyl include C₁₋₈alkyl, for example methyl, ethyl, n-propyl, iso-propyl, n-butyl, sec-butyl, iso-butyl, tert-butyl, pentyl, hexyl, 1,1-dimethylethyl, cyclopentyl or cyclohexyl or combinations thereof.

The term “alkoxy” as a group or as part of a group means a straight, branched or cyclic chain alkoxy group. Unless hereinbefore defined examples of alkoxy include C₁₋₈alkoxy, for example methoxy, ethoxy, n-propoxy, iso-propoxy, n-butoxy, sec-butoxy, iso-butoxy, tert-butoxy, pentoxy, hexyloxy, cyclopropoxy, cyclobutoxy, cyclopentoxy or cyclohexyloxy.

The term “alkenyl” means linear or branched structures and combinations thereof, of the indicated number of carbon atoms, having at least one carbon-to-carbon double bond, wherein hydrogen may be replaced by an additional carbon to carbon double bond. C₂₋₈alkenyl, for example, includes ethenyl, propenyl, 1-methylethenyl, butenyl and the like.

The term “alkynyl” means linear or branched structures and combinations thereof, of the indicated number of carbon atoms, having at least one carbon-to-carbon triple bond. C₂₋₈alkynyl, for example, includes ethynyl, propynyl, butynyl and the like.

The term “heterocyclyl” as a group or as part of a group means an aromatic or non-aromatic five or six membered ring which contains from 1 to 4 heteroatoms selected from nitrogen, oxygen or sulfur and unsubstituted or substituted by, for example, up to three substituents, preferably one or two substituents. Examples of 5-membered heterocyclyl groups include furan, tetrahydrofuran, thiophene, tetrahydrothiophene, pyrrole, pyrroline, pyrrolidine, dioxolane, oxazole, thiazole, imidazole, imidazoline, imidazolidine, pyrazole, pyrazoline, pyrazolidine, isoxazole, isothiazole, oxadiazole, triazole, thiadiazole, and tetrazole. Examples of 6-membered heterocyclyl groups include pyran, tetrahydropyran, pyridine, piperidine, dioxane, morpholine, dithiane, thiomorpholine, pyridazine, pyrimidine, pyrazine, piperazine, and triazine.

The term “bicyclic heterocyclyl” when used herein means a fused bicyclic aromatic or non-aromatic bicyclic heterocyclyl ring system comprising up to four, preferably one or two, heteroatoms each selected from oxygen, nitrogen and sulphur. Each ring may have from 4 to 7, preferably 5 or 6, ring atoms. A bicyclic heteroaromatic ring system may include a carbocyclic ring. Examples of bicyclic heterocyclyl groups include quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl or naphthyridinyl.

The term “aryl” as a group or as part of a group means a 5- or 6-membered aromatic ring for example phenyl, or a 7 to 12 membered bicyclic ring system where at least one of the rings is aromatic, for example naphthyl. An aryl group may be substituted by up to four, preferably one to three substituents. Preferably the aryl group is phenyl.

The term “heteroaryl” as a group or as part of a group means a monocyclic five or six membered aromatic ring, or a fused bicyclic aromatic ring system comprising two of such monocyclic five or six membered aromatic rings. These heteroaryl rings contain one or more heteroatoms selected from nitrogen, oxygen or sulfur, where N-oxides, sulfur oxides and sulfur dioxides are permissible heteroatom substitutions. A heteroaryl group may be optionally substituted by one or more substituents for example one or two substituents. Examples of “heteroaryl” used herein include furyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyrimidinyl, quinolinyl, isoquinolinyl, benzofuryl, benzothienyl, indolyl, and indazolyl.

Optional substituents for alkyl, alkenyl or alkynyl groups unless hereinbefore defined include OH, CO₂H, CO₂C₁₋₆alkyl, NHC₁₋₆alkyl, NH₂, (O), OC₁₋₆alkyl, phenyl or halo e.g. Cl, Br or F. An alkyl, alkenyl or alkynyl group may be substituted by one or more optional substituents, for example up to 5, 4, 3, 2 or 1 optional substituents. Particular substituted alkyl groups include those substituted by one or more fluorine atoms, up to per-fluorination, e.g. CH₂F, CHF₂, CF₃, C₂F₅, CH₂CF₃, and CH₂CH₂CF₃. Particular alkynyl substituents include phenyl.

Optional substituents for alkoxy groups unless hereinbefore defined include OH, and halo e.g. Cl, Br or F. An alkoxy group may be substituted by one or more optional substituents, for example up to 5, 4, 3, or 2 optional substituents. Particular substituted alkoxy groups include those subsituted by one or more fluorines e.g. OCH₂F, OCHF₂, OCF₃, OC₂F₅ etc.

Optional substituents for aryl, heteroaryl or heterocyclyl groups, unless hereinbefore defined, include one or two substituents selected from halogen; C₁₋₆alkyl; and C₁₋₆alkoxy.

When the heteroatom nitrogen replaces a carbon atom in a C₁₋₈alkyl group, or when nitrogen is present in a heteroaryl, heterocyclyl or bicyclic heterocyclyl group the nitrogen atom will, where appropriate be substituted by one or two substituents selected from hydrogen and C₁₋₈alkyl, preferably hydrogen and C₁₋₈alkyl, more preferably hydrogen.

Compounds of formula (I) can be prepared as set forth in the following scheme and in the examples. The following processes form another aspect of the present invention: 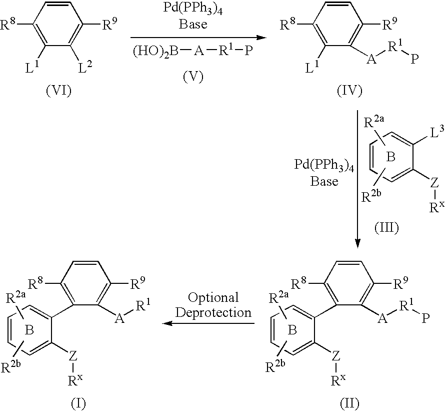
wherein L¹ and L² each represent a leaving group for example halo, e.g. bromo or iodo; L³ is an activating group, e.g. a boronic acid, P is an optional protecting group for example methyl, ethyl or benzyl esters; and A, B, R^{2a}, R^{2b}, Z, R⁸, R⁹, R¹ and R^{x} are as defined for compounds of formula (I).

The skilled person will recognise when the use of a protecting group is necessary.

When R¹ is CO₂H examples of protecting groups include C₁₋₄alkyl, e.g. methyl, ethyl, or benzyl esters.

Suitable reaction conditions for the deprotection of a compound of formula (II) include heating in ethanolic sodium hydroxide solution.

Suitable reaction conditions for the reaction of a compound of formula (VI) with a boronic acid of formula (V), or a compound of formula (IV) with a compound of formula (III) include heating the intermediates with tetrakis(triphenylphosphine)palladium (0) and an inorganic base, for example potassium carbonate or silver carbonate, in a solvent, e.g. ethylene glycol dimethyl ether (DME), toluene and ethanol, preferably in a ratio of 1:1.

Accordingly the present invention also provides a process for the preparation of a compound of formula (I) or a derivative thereof: 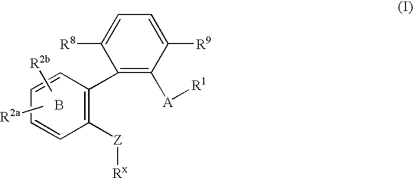
wherein:
A is an optionally substituted aryl, or an optionally substituted 5- or 6-membered heterocyclyl ring, or an optionally substituted bicyclic heterocyclyl group;
B is a phenyl or pyridyl ring;
Z is O, S, SO, or SO₂;
R¹ is CO₂H, CN, CONR⁵R⁶, CH₂CO₂R⁴, SO₂NR⁵R⁶, NR⁴CONR⁵R⁶, COalkyl, 2H-tetrazol-5-yl-methyl, optionally substituted SO₂alkyl, optionally substituted bicyclic heterocycle or optionally substituted heterocyclyl;
R^{2a} and R^{2b} are each independently selected from hydrogen, halogen, CONR⁵R⁶, CN, SO₂alkyl, SR⁵, NO₂, optionally substituted alkyl, optionally substituted alkoxy, optionally substituted aryl, and optionally substituted heteroaryl;
R^{x} is optionally substituted alkyl wherein 1 or 2 of the non-terminal carbon atoms are optionally replaced by a group independently selected from NR⁴, O and SOₙ, wherein n is 0, 1 or 2; optionally substituted alkenyl; or optionally substituted alkynyl: or R^{x} is optionally substituted CQ^{a}Q^{b}-heterocyclyl; optionally substituted CQ^{a}Q^{b}-bicyclic heterocyclyl; or optionally substituted CQ^{a}Q^{b}-aryl;
R⁴ is hydrogen or an optionally substituted alkyl;
R⁵ is hydrogen or an optionally substituted alkyl;
R⁶ is hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ is optionally substituted alkyl, optionally substituted heteroaryl or optionally substituted aryl;
R⁸ and R⁹ are each independently selected from hydrogen, halogen, CF₃, C₁₋₃alkoxy and C₁₋₃alkyl;
Q^{a} and Q^{b} are each independently selected from hydrogen and CH₃;
   wherein:
when A is a 6-membered ring the R¹ substituent and the above-defined 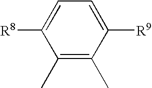
group are attached to carbon atoms 1,2-, or 1,3- relative to each other, and when A is a five-membered ring or bicyclic heterocyclyl group the R¹ substituent and the above-defined 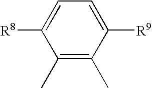
group are attached to substitutable carbon atoms 1,2- or 1,3- relative to each other; and
neither R^{2a} nor R^{2b} are ortho to the Z substituent; and
R^{x} is not 4-(tert-butyl)benzyl or 4-methylpent-1-yl; and
when R¹ is CONH₂, it is ortho to the 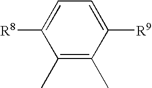
group; and
when R¹ is SO₂NR⁵R⁶, then R⁵ is hydrogen and R⁶ is COR⁷;
   comprising:
   reacting a compound of formula (IV): 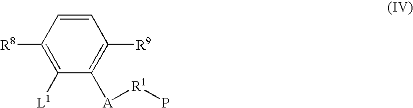
wherein R⁸, R⁹, A, and R¹ are as hereinbefore defined above for a compound of formula (I), L¹ is a leaving group and P is an optional protecting group;
   with a compound of formula (III): 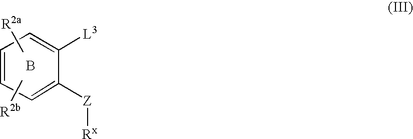
wherein L³ is an activating group R^{2a}, R^{2b}, B, Z, and R^{x} are as hereinbefore defined above for a compound of formula (I);
   and where required carrying out the following optional steps in any order:
converting one group R^{x} to another group R^{x}; and/or
effecting deprotection; and/or
converting one group R¹ to another group R¹; and/or
forming a derivative of the compound of formula (I) so formed:
   providing that the compound of Formula (I) is not:
2-benzyloxy-5-chloro-[1,1′;2′,1″]terphenyl-3″-carboxylic acid;
(2-benzyloxy-5-chloro-[1,1′;2′,1″]terphenyl-3″-yl)-acetic acid;
(2-benzyloxy-5-chloro[1,1′;2′,1″]terphenyl-2″-yl)acetic acid;
(2-benzyloxy-5-chloro[1,1′;2′,1″]terphenyl-4″-yl)acetic acid;
5″-acetylamino-2-benzyloxy-5-chloro[1,1′;2′,1″]terphenyl-3″-carboxylic acid;
2-benzyloxy-5-chloro-5″-propionylamino[1,1′;2′1″]terphenyl-3″-carboxylic acid;
2-benzyloxy-5-chloro-5″-(2-methylpropanoylamino)-[1,1′;2′,1″]terphenyl-3″-carboxylic acid;
2-benzoyloxy-5″-butyrylamino-5-chloro[1,1′;2′,1″]terphenyl-3″-carboxylic acid;
2-benzyloxy-5-chloro-5″-[(1-phenyl-methanoyl)amino]-[1,1′;2′,1″]terphenyl-3″-carboxylic acid;
2-benzyloxy-5-chloro-5″-methanesulfonylamino-[1,1′;2′,1″]terphenyl-3″-carboxylic acid
5″-amino-2-benzyloxy-5-chloro[1,1′;2′,2″]-3″-carboxylic acid;
2-benzyloxy-5″-butyrylamino-5-trifluoromethyl[1,1′;2′,1″]terphenyl-3″-carboxylic acid-3-carboxylic acid;
2-benzyloxy-4″-chloro[1,1′;2′,1″]terphenyl 2″-carboxylic acid;
2-benzyloxy-5″-fluoro-[1,1′;2′,1″]terphenyl-2″-carboxylic acid;
2-benzyloxy-4″-fluoro-[1,1′;2′,1″]terphenyl-2″-carboxylic acid;
2″-benzyloxy-5-fluoro-[1,1′;2′,1″]terphenyl-3-carboxylic acid;
4″-amino-2-benzyloxy-[1,1′;2′,1″]terphenyl-3″-carboxylic acid;
5″-acetylamino-2-benzyloxy-[1,1′;2′,1″]terphenyl-2″-carboxylic acid;
2-benzyloxy-5-chloro-[1,1′;2′,1″]terphenyl-2″-carboxylic acid;
2-benzyloxy-[1,1′;2′,1″]terphenyl-3″-carboxylic acid;
2-benzyloxy-5-chloro-[1,1′;2′,1″]terphenyl-2″-carboxylic acid amide;
5-(2-benzyloxy-5-chloro-[1,1′;2′,1″]terphenyl-3″-yl)-1H-tetrazole;
N-[1-(2-benzyloxy-5-chloro-[1,1′;2′,1″]terphenyl-2″-yl)-methanoyl]-benzenesulfonamide;
2-benzyloxy-[1,1′;2′,1″]terphenyl-4″-sulfonic acid (1-phenyl-methanoyl)-amide;
2-benzyloxy-[1,1′;2′,1″]terphenyl-4″-sulfonic acid [1-(4-nitro-phenyl)-methanoyl]-amide;
2-benzyloxy-[1,1′;2′,1″]terphenyl-3″-sulfonic acid acetyl-amide;
5-chloro-2-(3-methyl-butoxy)-[1,1′;2′,1″]terphenyl-3″-carboxylic acid;
5-chloro-2-(4-fluoro-benzyloxy)-[1,1′;2′,1″]terphenyl-3″-carboxylic acid;
5-chloro-2-(2,4-difluoro-benzyloxy)-[1,1′;2′,1″]terphenyl-3″-carboxylic acid;
5-chloro-2-(4-chloro-benzyloxy)-[1,1′;2′,1″]terphenyl-3″carboxylic acid;
5-chloro-2-(2-fluoro-4-chloro-benzyloxy)-[1,1′;2′,1″]terphenyl-3″carboxylic;
5-chloro-2-(4-isobutoxy)-[1,1′,2′,1″]terphenyl-3″-carboxylic acid;
5-chloro-2-(pyridin-2-ylmethoxy)-[1,1′;2′,1″]terphenyl-3″carboxylic acid;
5-chloro-2-(pyridin-4-ylmethoxy)-[1,1′;2′,1″]terphenyl-3″carboxylic acid;
5-chloro-2-(pyridin-3-ylmethoxy)-[1,1′;2′,1″]terphenyl-3″carboxylic acid;
5-chloro-2-cyclohexylmethoxy-[1,1′;2′,1″]terphenyl-3″carboxylic acid;
5-chloro-2-(thiophen-3-ylmethoxy)-[1,1′;2′,1″]terphenyl-3″carboxylic acid;
5-chloro-2-(thiophen-2-ylmethoxy)-[1,1′;2′,1″]terphenyl-3″carboxylic acid;
5-chloro-2-cyclopentylmethoxy-[1,1′;2′,1″]terphenyl-3″carboxylic acid;
5-chloro-2-propoxy-[1,1′;2′,1″]terphenyl-3″-carboxylic acid;
2-butoxy-5-chloro-[1,1′;2′,1″]terphenyl-3″-carboxylic acid;
5-chloro-2-isopropoxy-[1,1′;2′,1″]terphenyl-3″-carboxylic acid;
5-chloro-2-isobutoxy-[1,1′;2′,1″]terphenyl-2″-carboxylic acid;
2″-{[(4-fluorophenyl)methyl]oxy}-5-[(methyloxy)carbonyl]-5″-(trifluoromethyl)-1,1′:2′,1″-terphenyl-3-carboxylic acid;
5-chloro-2″-[(phenylmethyl)oxy]-1,1′:2′,1″-terphenyl-2-carboxylic acid;
4-(methoxy)-2″-[(phenylmethyl)oxy]-1,1′:2′,1″-terphenyl-2-carboxylic acid;
2″-{[(2,4-difluorophenyl)methyl]oxy}-4-(propanoylamino)-5″-(trifluoromethyl)-1,1′:2′,1″-terphenyl-2-carboxylic acid;
2″-{[(2,4-difluorophenyl)methyl]oxy}-4-[(2-methylpropanoyl)amino]-5″-(trifluoromethyl)-1,1′:2′,1″-terphenyl-2-carboxylic acid;
5-(2-oxo-1-pyrrolidinyl)-2″-[(phenylmethyl)oxy]-5″-(trifluoromethyl)-1,1′:2′,1″-terphenyl-3-carboxylic acid;
2″-{[(4-fluorophenyl)methyl]oxy}-5″-(trifluoromethyl)-1,1′:2′,1″-terphenyl-3,5-dicarboxylic acid;
2″-{[(4-fluorophenyl)methyl]oxy}-5-{[(2-methylpropyl)amino]carbonyl}-5″-(trifluoromethyl)-1,1′:2′,1″-terphenyl-3-carboxylic acid;
6-[2′-{[(4-fluorophenyl)methyl]oxy}-5′-(trifluoromethyl)-2-biphenylyl]-2-pyrazinecarboxylic acid;
2″-{[(4-fluorophenyl)methyl]oxy}-5-(propanoylamino)-5″-(trifluoromethyl)-1,1′:2′,1″-terphenyl-3-carboxylic acid;
2″-[(phenylmethyl)oxy]-5-(propanoylamino)-5″-(trifluoromethyl)-1,1′:2′,1″-terphenyl-3-carboxylic acid;
2″-{[(2,4-difluorophenyl)methyl]oxy}-5-(propanoylamino)-5″-(trifluoromethyl)-1,1′:2′,1″-terphenyl-3-carboxylic acid;
5″-chloro-5-{[(methyloxy)acetyl]amino}-2″-[(phenylmethyl)oxy]-1,1′:2′,1″-terphenyl-3-carboxylic acid;
5″-chloro-2″-[(phenylmethyl)oxy]-5-[(2-thienylacetyl)amino]-1,1′:2′,1″-terphenyl-3-carboxylic acid;
5″-chloro-2″-[(phenylmethyl)oxy]-5-({[(phenylmethyl)oxy]acetyl}amino)-1,1′:2′,1″-terphenyl-3-carboxylic acid;
5-{[(1-acetyl-4-piperidinyl)carbonyl]amino}-5″-chloro-2″-[(phenylmethyl)oxy]-1,1′:2′,1″-terphenyl-3-carboxylic acid;
5″-chloro-5-[(phenylacetyl)amino]-2″-[(phenylmethyl)oxy]-1,1′:2′,1″-terphenyl-3-carboxylic acid;
5″-chloro-5-{[(3,5-dimethyl-4-isoxazolyl)carbonyl]amino}-2″-[(phenylmethyl)oxy]-1,1′:2′,1″-terphenyl-3-carboxylic acid;
5″-chloro-5-[(3-methylbutanoyl)amino]-2″-[(phenylmethyl)oxy]-1,1′:2′,1″-terphenyl-3-carboxylic acid;
5″-chloro-5-(glycylamino)-2″-[(phenylmethyl)oxy]-1,1′:2′,1″-terphenyl-3-carboxylic acid;
2″-[(penylmethyl)oxy]-4-(propanoylamino)-1,1′:2′,1″-terphenyl-2-carboxylic acid;
4-[(2-methylpropanoyl)amino]-2″-[(phenylmethyl)oxy]-1,1′:2′,1″-terphenyl-2-carboxylic acid;
5-cyano-2″-{[(2,4-difluorophenyl)methyl]oxy}-5″-(trifluoromethyl)-1,1′:2′,1″-terphenyl-3-carboxylic acid;
5″-bromo-5-cyano-2″-[(phenylmethyl)oxy]-1,1′:2′,1″-terphenyl-3-carboxylic acid;
5-cyano-2″-[(phenylmethyl)oxy]-5″-(trifluoromethyl)-1,1′:2′,1″-terphenyl-3-carboxylic acid;
5-(aminocarbonyl)-2″-{[(4-fluorophenyl)methyl]oxy}-5″-(trifluoromethyl)-1,1′:2′,1″-terphenyl-3-carboxylic acid;
2″-{[(4-fluorophenyl)methyl]oxy}-5-{[(2-hydroxyethyl)amino]carbonyl}-5″-(trifluoromethyl)-1,1′:2′,1″-terphenyl-3-carboxylic acid;
2″-{[(4-fluorophenyl)methyl]oxy}-5-{[(3-pyridinylmethyl)amino]carbonyl}-5″-(trifluoromethyl)-1,1′:2′,1″-terphenyl-3-carboxylic acid;
6-{5′-chloro-2′-[(phenylmethyl)oxy]-2-biphenylyl}-2-pyridinecarboxylic acid;
6-(5′-chloro-2′-{[(4-fluorophenyl)methyl]oxy}-2-biphenylyl)-2-pyridinecarboxylic acid;
6-(5′-chloro-2′-{[(2,4-difluorophenyl)methyl]oxy}-2-biphenylyl)-2-pyridinecarboxylic acid;
2-[2′-{[(4-fluorophenyl)methyl]oxy}-5′-(trifluoromethyl)-2-biphenylyl]-4-pyridinecarboxylic acid;
3-amino-6-[2′-{[(4-fluorophenyl)methyl]oxy}-5′-(trifluoromethyl)-2-biphenylyl]-2-pyrazinecarboxylic acid;
4-(acetylamino)-2″-[(phenylmethyl)oxy]-5″-(trifluoromethyl)-1,1′:2′,1″-terphenyl-2-carboxylic acid;
4-(acetylamino)-2″-{[(4-fluorophenyl)methyl]oxy}-5″-(trifluoromethyl)-1,1′:2′,1″-terphenyl-2-carboxylic acid;
4-(acetylamino)-2″-{[(2,4-difluorophenyl)methyl]oxy}-5″-(trifluoromethyl)-1,1′:2′,1″-terphenyl-2-carboxylic acid;
4-methyl-2″-[(phenylmethyl)oxy]-1,1′:2′,1″-terphenyl-2-carboxylic acid;
2″-{[(2,4-difluorophenyl)methyl]oxy}-4-methyl-5″-(trifluoromethyl)-1,1′:2′,1″-terphenyl-2-carboxylic acid;
2-benzyloxy-5-fluoro[1,1′,2,2′]terphenyl-3″-carboxylic acid;
2-(4-fluorobenzyl)oxy-5-fluoro[1,1′,2,2′]terphenyl-3″-carboxylic acid;
2-(2,4-difluorobenzyl)oxy-5-fluoro[1,1′,2,2′]terphenyl-3″-carboxylic acid;
2′-{5-chloro-2-(benzyloxy)-3-pyridinyl]-3-biphenylcarboxylic acid;
2′-{6-chloro-3-[(phenylmethyl)oxy]-2-pyridinyl}-3-biphenylcarboxylic acid;
5-amino-2′-{6-chloro-3-[(phenylmethyl)oxy]-2-pyridinyl}-3-biphenylcarboxylic acid;
5-{2′-[(phenylmethyl)oxy]-2-biphenylyl}-3-pyridinecarboxylic acid;
4″-chloro-2″-[(phenylmethyl)oxy]-1,1′:2′,1″-terphenyl-2-carboxylic acid; or
6″-fluoro-2-benzyloxy-[1,1′;2′,1″]terphenyl-3″-carboxylic acid.

Alternatively the compounds of formula (I) may be prepared by the route described below: 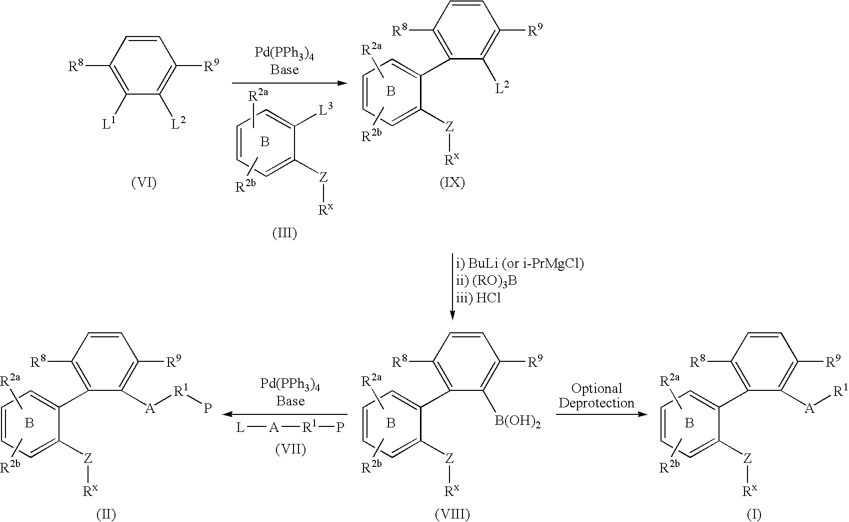
wherein L, L¹ and L² are each a leaving group for example halo, e.g. bromo or iodo; L³ is an activating group e.g. boronic acid, P is an optional protecting group, R is C₁₋₄ alkyl e.g. methyl or isopropyl, and A, B, R^{2a}, R^{2b}, Z, R⁸, R⁹, R¹ and R^{x} are as defined for compounds of formula (I). L¹ may be converted to L^{1a} wherein L^{1a} is an activating group, e.g. a boronic acid, and in this situation L³ may be a leaving group e.g. halo.

When R^{2a} and/or R^{2b} are bromo then L should be bromo, L³ should be an activating group and L¹ and L² are each iodo.

When R¹ is CO₂H examples of protecting groups include C₁₋₄alkyl, e.g. methyl, ethyl, or benzyl esters.

Suitable reaction conditions for the deprotection of a compound of formula (II) include heating in ethanolic sodium hydroxide solution.

Suitable reaction conditions for the reaction of a compound of formula (VI) with a compound of formula (III), or a compound of formula (VII) with a boronic acid of formula (VIII) include heating with tetrakis(triphenylphosphine)palladium (0) and an inorganic base, for example potassium carbonate or silver carbonate, in a solvent, e.g. ethylene glycol dimethyl ether (DME), toluene and ethanol, preferably in a ratio of 1:1.

Suitable reaction conditions for the conversion of a compound of formula (IX) to a compound of formula (VIII) include reacting the compound of formula (IX) wherein L² is Br or I with butyl lithium (BuLi) or iso-propyl magnesium chloride in a solvent such as diethyl ether or tetrahydrofuran, treating with trimethyl borate and subsequent acidification.

Accordingly the present invention also provides a process for the preparation of a compound of formula (I) or a derivative thereof: 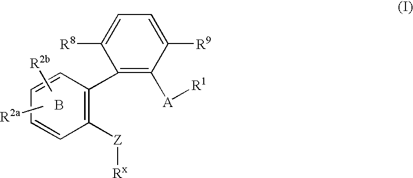
wherein:
A is an optionally substituted aryl, or an optionally substituted 5- or 6-membered heterocyclyl ring, or an optionally substituted bicyclic heterocyclyl group;
B is a phenyl or pyridyl ring;
Z is O, S, SO, or SO₂;
R¹ is CO₂H, CN, CONR⁵R⁶, CH₂CO₂R⁴, SO₂NR⁵R⁶, NR⁴CONR⁵R⁶, COalkyl, 2H-tetrazol-5-yl-methyl, optionally substituted SO₂alkyl, optionally substituted bicyclic heterocycle or optionally substituted heterocyclyl;
R^{2a} and R^{2b} are each independently selected from hydrogen, halogen, CONR⁵R⁶, CN, SO₂alkyl, SR⁵, NO₂, optionally substituted alkyl, optionally substituted alkoxy, optionally substituted aryl, and optionally substituted heteroaryl;
R^{x} is optionally substituted alkyl wherein 1 or 2 of the non-terminal carbon atoms are optionally replaced by a group independently selected from NR⁴, O and SOₙ, wherein n is 0, 1 or 2; optionally substituted alkenyl; or optionally substituted alkynyl: or R^{x} is optionally substituted CQ^{a}Q^{b}-heterocyclyl; optionally substituted CQ^{a}Q^{b}-bicyclic heterocyclyl; or optionally substituted CQ^{a}Q^{b}-aryl;
R⁴ is hydrogen or an optionally substituted alkyl;
R⁵ is hydrogen or an optionally substituted alkyl;
R⁶ is hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ is optionally substituted alkyl, optionally substituted heteroaryl or optionally substituted aryl;
R⁸ and R⁹ are each independently selected from hydrogen, halogen, CF₃, C₁₋₃alkoxy and C₁₋₃alkyl;
Q^{a} and Q^{b} are each independently selected from hydrogen and CH₃;
   wherein:
when A is a 6-membered ring the R¹ substituent and the above-defined 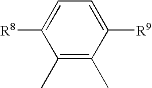
group are attached to carbon atoms 1,2-, or 1,3- relative to each other, and when A is a five-membered ring or bicyclic heterocyclyl group the R¹ substituent and the above-defined 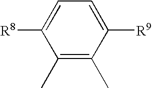
group are attached to substitutable carbon atoms 1,2- or 1,3- relative to each other; and
neither R^{2a} nor R^{2b} are ortho to the Z substituent; and
R^{x} is not 4-(tert-butyl)benzyl or 4-methylpent-1-yl; and
when R¹ is CONH₂, it is ortho to the 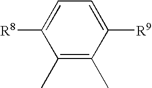
group; and
when R¹ is SO₂NR⁵R⁶, then R⁵ is hydrogen and R⁶ is COR⁷;
   comprising:
   reacting a compound of formula (VII): 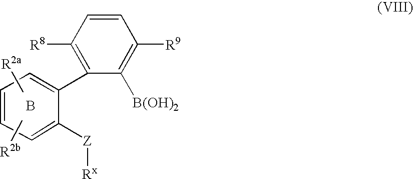
   wherein B, R^{2a}, R^{2b}, Z, R⁸, R⁹, and R^{x} are as defined for compounds of formula (I), with a compound of formula (VII):
   L-A-R¹P (VII)
   wherein A and R¹ are as hereinbefore defined for compounds of formula (I), L is a leaving group and P is an optional protecting group;
   and where required carrying out the following optional steps in any order:
converting one group R^{x} to another group R^{x}; and/or
effecting deprotection; and/or
converting one group R¹ to another group R¹; and/or
forming a derivative of the compound of formula (I) so formed;
   provided that the compound of Formula (I) is not:
2-benzyloxy-5-chloro-[1,1′;2′,1″]terphenyl-3″-carboxylic acid;
(2-benzyloxy-5-chloro-[1,1′;2′,1″]terphenyl-3″-yl)-acetic acid;
(2-benzyloxy-5-chloro[1,1′;2′,1″]terphenyl-2″-yl)acetic acid;
(2-benzyloxy-5-chloro[1,1′;2′,1″]terphenyl-4″-yl)acetic acid;
5″-acetylamino-2-benzyloxy-5-chloro[1,1′;2′,1″]terphenyl-3″-carboxylic acid;
2-benzyloxy-5-chloro-5″-propionylamino[1,1′;2′,1″]terphenyl-3″-carboxylic acid;
2-benzyloxy-5-chloro-5″-(2-methylpropanoylamino)-[1,1′;2′,1″]terphenyl-3″-carboxylic acid;
2-benzoyloxy-5″-butyrylamino-5-chloro[1,1′;2′,1″]terphenyl-3″-carboxylic acid;
2-benzyloxy-5-chloro-5″-[(1-phenyl-methanoyl)amino]-[1,1′;2′,1″]terphenyl-3″-carboxylic acid;
2-benzyloxy-5-chloro-5″-methanesulfonylamino-[1,1′;2′,1″]terphenyl-3″-carboxylic acid
5″-amino-2-benzyloxy-5-chloro[1,1′;2′,2″]-3″-carboxylic acid;
2-benzyloxy-5″-butyrylamino-5-trifluoromethyl[1,1′;2′,1″]terphenyl-3″-carboxylic acid-3-carboxylic acid;
2-benzyloxy-4″-chloro[1,1′;2′,1″]terphenyl 2″-carboxylic acid;
2-benzyloxy-5″-fluoro-[1,1′;2′,1″]terphenyl-2″-carboxylic acid;
2-benzyloxy-4″-fluoro-[1,1′;2′,1″]terphenyl-2″-carboxylic acid;
2″-benzyloxy-5-fluoro-[1,1′;2′,1″]terphenyl-3-carboxylic acid;
4″-amino-2-benzyloxy-[1,1′;2′,1″]terphenyl-3″-carboxylic acid;
5″-acetylamino-2-benzyloxy-[1,1′;2′,1″]terphenyl-2″-carboxylic acid;
2-benzyloxy-5-chloro-[1,1′;2′,1″]terphenyl-2″-carboxylic acid;
2-benzyloxy-[1,1′;2′,1″]terphenyl-3″-carboxylic acid;
2-benzyloxy-5-chloro-[1,1′;2′,1″]terphenyl-2″-carboxylic acid amide;
5-(2-benzyloxy-5-chloro-[1,1′;2′,1″]terphenyl-3″-yl)-1H-tetrazole;
N-[1-(2-benzyloxy-5-chloro-[1,1′;2′,1″]terphenyl-2″-yl)-methanoyl]-benzenesulfonamide;
2-benzyloxy-[1,1′;2′,1″]terphenyl-4″-sulfonic acid (1-phenyl-methanoyl)-amide;
2-benzyloxy-[1,1′;2′,1″]terphenyl-4″-sulfonic acid [1-(4-nitro-phenyl)-methanoyl]-amide;
2-benzyloxy-[1,1′;2′,1″]terphenyl-3″-sulfonic acid acetyl-amide;
5-chloro-2-(3-methyl-butoxy)-[1,1′;2′,1″]terphenyl-3″-carboxylic acid;
5-chloro-2-(4-fluoro-benzyloxy)-[1,1′;2′,1″]terphenyl-3″-carboxylic acid;
5-chloro-2-(2,4-difluoro-benzyloxy)-[1,1′;2′,1″]terphenyl-3″-carboxylic acid;
5-chloro-2-(4-chloro-benzyloxy)-[1,1′;2′,1″]terphenyl-3″carboxylic acid;
5-chloro-2-(2-fluoro-4-chloro-benzyloxy)-[1,1′;2′,1″]terphenyl-3″carboxylic;
5-chloro-2-(4-isobutoxy)-[11′,2′,1″]terphenyl-3″-carboxylic acid;
5-chloro-2-(pyridin-2-ylmethoxy)-[1,1′;2′,1″]terphenyl-3″carboxylic acid;
5-chloro-2-(pyridin-4-ylmethoxy)-[1,1′;2′,1″]terphenyl-3″carboxylic acid;
5-chloro-2-(pyridin-3-ylmethoxy)-[1,1′;2′,1″]terphenyl-3″carboxylic acid;
5-chloro-2-cyclohexylmethoxy-[1,1′;2′,1″]terphenyl-3″carboxylic acid;
5-chloro-2-(thiophen-3-ylmethoxy)-[1,1′;2′,1″]terphenyl-3″carboxylic acid;
5-chloro-2-(thiophen-2-ylmethoxy)-[1,1′;2′,1″]terphenyl-3″carboxylic acid;
5-chloro-2-cyclopentylmethoxy-[1,1′;2′,1″]terphenyl-3″carboxylic acid;
5-chloro-2-propoxy-[1,1′;2′,1″]terphenyl-3″-carboxylic acid;
2-butoxy-5-chloro-[1,1′;2′,1″]terphenyl-3″-carboxylic acid;
5-chloro-2-isopropoxy-[1,1′;2′,1″]terphenyl-3″-carboxylic acid;
5-chloro-2-isobutoxy-[1,1′;2′,1″]terphenyl-2″-carboxylic acid;
2″-{[(4-fluorophenyl)methyl]oxy}-5-[(methyloxy)carbonyl]-5″-(trifluoromethyl)-1,1′:2′,1″-terphenyl-3-carboxylic acid;
5-chloro-2″-[(phenylmethyl)oxy]-1,1′:2′,1″-terphenyl-2-carboxylic acid;
4-(methoxy)-2″-[(phenylmethyl)oxy]-1,1′:2′,1″-terphenyl-2-carboxylic acid;
2″-{[(2,4-difluorophenyl)methyl]oxy}-4-(propanoylamino)-5″-(trifluoromethyl)-1,1′:2′,1″-terphenyl-2-carboxylic acid;
2″-{[(2,4-difluorophenyl)methyl]oxy}-4-[(2-methylpropanoyl)amino]-5″-(trifluoromethyl)-1,1′:2′,1″-terphenyl-2-carboxylic acid;
5-(2-oxo-1-pyrrolidinyl)-2″-[(phenylmethyl)oxy]-5″-(trifluoromethyl)-1,1′:2′,1″-terphenyl-3-carboxylic acid;
2″-{[(4-fluorophenyl)methyl]oxy}-5″-(trifluoromethyl)-1,1′:2′,1″-terphenyl-3,5-dicarboxylic acid;
2″-{[(4-fluorophenyl)methyl]oxy}-5-{[(2-methylpropyl)amino]carbonyl}-5″-(trifluoromethyl)-1,1′:2′,1″-terphenyl-3-carboxylic acid;
6-[2′-{[(4-fluorophenyl)methyl]oxy}-5′-(trifluoromethyl)-2-biphenylyl]-2-pyrazinecarboxylic acid;
2″-{[(4-fluorophenyl)methyl]oxy}-5-(propanoylamino)-5″-(trifluoromethyl)-1,1′:2′,1″-terphenyl-3-carboxylic acid;
2″-[(phenylmethyl)oxy]-5-(propanoylamino)-5″-(trifluoromethyl)-1,1′:2′,1″-terphenyl-3-carboxylic acid;
2″-{[(2,4-difluorophenyl)methyl]oxy}-5-(propanoylamino)-5″-(trifluoromethyl)-1,1′:2′,1″-terphenyl-3-carboxylic acid;
5″-chloro-5-{[(methyloxy)acetyl]amino}-2″-[(phenylmethyl)oxy]-1,1′:2′,1″-terphenyl-3-carboxylic acid;
5″-chloro-2″-[(phenylmethyl)oxy]-5-[(2-thienylacetyl)amino]-1,1′:2′,1″-terphenyl-3-carboxylic acid;
5″-chloro-2″-[(phenylmethyl)oxy]-5-({[(phenylmethyl)oxy]acetyl}amino)-1,1′:2′,1″-terphenyl-3-carboxylic acid;
5-{[(1-acetyl-4-piperidinyl)carbonyl]amino}-5″-chloro-2″-[(phenylmethyl)oxy]-1,1′:2′,1″-terphenyl-3-carboxylic acid;
5″-chloro-5-[(phenylacetyl)amino]-2″-[(phenylmethyl)oxy]-1,1′:2′,1″-terphenyl-3-carboxylic acid;
5″-chloro-5-{[(3,5-dimethyl-4-isoxazolyl)carbonyl]amino}-2″-[(phenymethyl)oxy-1,1′:2′,1″-terphenyl-3-carboxylic acid;
5″-chloro-5-[(3-methylbutanoyl)amino]-2″-[(phenylmethyl)oxy]-1,1′:2′,1″-terphenyl-3-carboxylic acid;
5″-chloro-5-(glycylamino)-2″-[(phenylmethyl)oxy]-1,1′:2′,1″-terphenyl-3-carboxylic acid;
2″-[(penylmethyl)oxy]-4-(propanoylamino)-1,1′:2′,1″-terphenyl-2-carboxylic acid;
4-[(2-methylpropanoyl)amino]-2″-[(phenylmethyl)oxy]-1,1′:2′,1″-terphenyl-2-carboxylic acid;
5-cyano-2″-{[(2,4-difluorophenyl)methyl]oxy}-5″-(trifluoromethyl)-1,1′:2′,1″-terphenyl-3-carboxylic acid;
5″-bromo-5-cyano-2″-[(phenylmethyl)oxy]-1,1′:2′,1″-terphenyl-3-carboxylic acid;
5-cyano-2″-[(phenylmethyl)oxy]-5″-(trifluoromethyl)-1,1′:2′,1″-terphenyl-3-carboxylic acid;
5-(aminocarbonyl)-2″-{[(4-fluorophenyl)methyl]oxy}-5″-(trifluoromethyl)-1,1′:2′,1″-terphenyl-3-carboxylic acid;
2″-{[(4-fluorophenyl)methyl]oxy}-5-{[(2-hydroxyethyl)amino]carbonyl}-5″-(trifluoromethyl)-1,1′:2′,1″-terphenyl-3-carboxylic acid;
2″-{[(4-fluorophenyl)methyl]oxy}-5-{[(3-pyridinylmethyl)amino]carbonyl}-5″-(trifluoromethyl)-1,1′:2′,1″-terphenyl-3-carboxylic acid;
6-{5′-chloro-2′-[(phenylmethyl)oxy]-2-biphenylyl}-2-pyridinecarboxylic acid;
6-(5′-chloro-2′-{[(4-fluorophenyl)methyl]oxy}-2-biphenylyl)-2-pyridinecarboxylic acid;
6-(5′-chloro-2′-{[(2,4-difluorophenyl)methyl]oxy}-2-biphenylyl)-2-pyridinecarboxylic acid;
2-[2′-{[(4-fluorophenyl)methyl]oxy}-5′-(trifluoromethyl)-2-biphenylyl]-4-pyridinecarboxylic acid;
3-amino-6-[2′-{[(4-fluorophenyl)methyl]oxy}-5′-(trifluoromethyl)-2-biphenylyl]-2-pyrazinecarboxylic acid;
4-(acetylamino)-2″-[(phenylmethyl)oxy]-5″-(trifluoromethyl)-1,1′:2′,1″-terphenyl-2-carboxylic acid;
4-(acetylamino)-2″-{[(4-fluorophenyl)methyl]oxy}-5″-(trifluoromethyl)-1,1′:2′,1″-terphenyl-2-carboxylic acid;
4-(acetylamino)-2″-{[(2,4-difluorophenyl)methyl]oxy}-5″-(trifluoromethyl)-1,1′:2′,1″-terphenyl-2-carboxylic acid;
4-methyl-2″-[(phenylmethyl)oxy]-1,1′:2′,1″-terphenyl-2-carboxylic acid;
2″-{[(2,4-difluorophenyl)methyl]oxy}-4-methyl-5″-(trifluoromethyl)-1,1′:2′,1″-terphenyl-2-carboxylic acid;
2-benzyloxy-5-fluoro[1,1′,2,2′]terphenyl-3″-carboxylic acid;
2-(4-fluorobenzyl)oxy-5-fluoro[1,1′,2,2′]terphenyl-3″-carboxylic acid;
2-(2,4-difluorobenzyl)oxy-5-fluoro[1,1′,2,2′]terphenyl-3″-carboxylic acid;
2′-{5-chloro-2-(benzyloxy)-3-pyridinyl]-3-biphenylcarboxylic acid;
2′-{6-chloro-3-[(phenylmethyl)oxy]-2-pyridinyl}-3-biphenylcarboxylic acid;
5-amino-2′-{6-chloro-3-[(phenylmethyl)oxy]-2-pyridinyl}-3-biphenylcarboxylic acid;
5-{2′-[(phenylmethyl)oxy]-2-biphenylyl}-3-pyridinecarboxylic acid;
4″-chloro-2″-[(phenylmethyl)oxy]-1,1′:2′,1″-terphenyl-2-carboxylic acid; or
6″-fluoro-2-benzyloxy-[1,1′;2′,1″]terphenyl-3″-carboxylic acid.

Suitable reaction conditions for the deprotection of a compound of formula (II) include heating in ethanolic sodium hydroxide solution.

It will be appreciated that certain substituents in intermediates and compounds of formula (I) may be converted to other substituents by conventional methods known to those skilled in the art.

A group R¹ may be converted to another group R¹ by use of conventional organic transformations known to those skilled in the art. For example R¹=CO₂H may be converted to an amide, e.g. CONHCQ^{a}Q^{b}aryl or CONHCQ^{a}Q^{b}heteroaryl wherein Q^{a} and Q^{b} are selected from hydrogen and CH₃, by conventional methods for the preparation of amides as described in, for example, Richard Larock, Comprehensive Organic Transformations, 2nd edition, Wiley-VCH, ISBN 0-471-19031-4.

Certain substituents in any of the reaction intermediates and compounds of formula (I) may be converted to other substituents by conventional methods known to those skilled in the art. Examples of substituents which may be converted include one group R^{x} to another group R^{x}; and one substituent on a group A to another substituent on a group A. Examples of such transformations include the reduction of a nitro group to give an amino group; alkylation and amidation of amino groups; hydrolysis of esters, alkylation of hydroxy and amino groups; and amidation and esterification of carboxylic acids. Such transformations are well known to those skilled in the art and are described in for example, Richard Larock, Comprehensive Organic Transformations, 2nd edition, Wiley-VCH, ISBN 0-471-19031-4.

For example, when R^{x} is p-methoxybenzyl, cleavage of the ether to give the phenol or pyridinol is carried out using, for example, using acid e.g. HCl/dioxane or HBr/acetic acid or using sodium methanethiolate. When R^{x} is methyl, cleavage of the ether to give the phenol is carried out using, for example, sodium methanethiolate. Cleavage of the ether to give a pyridinol is carried out in the presence of, for example, trifluoroacetic acid. Conversion to another R^{x} group, for example a substituted benzyl group, may be effected by reaction of the phenol or pyridinol with a suitable substituted benzyl bromide. The skilled person will appreciate that conversion of the protecting group P to another protecting group P may also occur under the reaction conditions used. When R^{x} is benzyl, cleavage of the ether to give the phenol or pyridinol may be carried out by hydrogenation according to known methods e.g. H₂—Pd/C or NH₄CO₂H—Pd/C. The resulting phenol or pyridinol can then be converted to another group R^{x} as described above.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

Phenyl intermediates of the formula (VI): 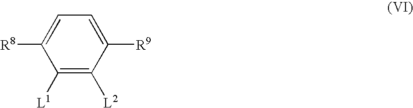
wherein L¹, L² are as defined above, and R⁸ and R⁹ are as hereinbefore defined for compounds of formula (I) are commercially available or may be readily prepared according to known methods. Compounds wherein L¹ is an activating group (L^{1a}) are commercially available or may be prepared from compounds wherein L¹ is a leaving group by conventional means.

Compounds of the formula (III): 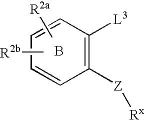
wherein L³ is as hereinbefore defined, R^{2a}, R^{2b}, Z, B and R^{x} and are as defined for compounds of formula (I) are commercially available, or may readily be prepared by methods known to those skilled in the art, for example from suitable commercially available pyridinols, anisoles or phenols or compounds of formula (X) using methods as described in the examples.

Intermediates of the formula (V):
(HO)₂B-A-R¹P
wherein P is an optional protecting group and R¹ and A are as hereinbefore defined for compounds of formula (I) are commercially available or may readily be prepared, for example, from suitable halobenzoic acid esters according to known methods, for example using methods as described in the examples.

Intermediates of the formula (VII):
L-A-R¹P
wherein L is a leaving group, e.g. Br, P is an optional protecting group and R¹ and A are as hereinbefore defined for compounds of formula (I) are commercially available or may readily be prepared according to known methods, for example using methods as described in the examples.

The preparation and reactions of boronic acids of formula (III), formula (V), formula (VIII) and (XI) is reviewed in Suzuki et al, Synth. Commun., 1981, 11, 513; Martin et al, Acta. Chim. Scand., 1993, 47, 221; and Miyaura et al, Chem. Rev., 1995, 95, 2457. For example, 2-benzyloxy-5-chlorophenylboronic acid may be prepared from 2-benzyloxy-5-chloro-iodobenzene. 2-Benzyloxy-5-chloro-iodobenzene may be prepared from 4-chloro-2-iodoanisole by demethylation followed by benzylation according to known methods.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The compounds of the invention bind to the EP₁ receptor and they are therefore considered to be useful in treating conditions mediated by the action of PGE₂ at EP₁ receptors.

Conditions mediated by the action of PGE₂ at EP₁ receptors include pain; fever; inflammation; immunological diseases; abnormal platelet function diseases; impotence or erectile dysfunction; bone disease; hemodynamic side effects of non-steroidal anti-inflammatory drugs; cardiovascular diseases; neurodegenerative diseases and neurodegeneration; neurodegeneration following trauma; tinnitus; dependence on a dependence-inducing agent; complications of Type I diabetes; and kidney dysfunction.

The compounds of formula (I) are considered to be useful as analgesics. They are therefore considered useful in the treatment or prevention of pain.

The compounds of formula (I) are considered useful as analgesics to treat acute pain, chronic pain, neuropatic pain, inflammatory pain, visceral pain, pain associated with cancer and fibromyalgia, pain associated with migraine, tension headache and cluster headaches, and pain associated with functional bowel disorders, non-cardiac chest pain and non-ulcer dispepsia.

The compounds of formula (I) are considered useful in the treatment of chronic articular pain (e.g. rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea. The compounds of the invention may also be considered useful in the treatment of visceral pain.

The compounds of the invention are considered to be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are incredibly heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous stimulation (dynamic, static or thermal allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

The compounds of formula (I) are also considered useful in the treatment of fever.

The compounds of formula (I) are also considered useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fancier's disease, farmer's lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

The compounds of formula (I) are also considered useful in the treatment of immunological diseases such as autoimmune diseases, immunological deficiency diseases or organ transplantation. The compounds of formula (I) are also effective in increasing the latency of HIV infection.

The compounds of formula (I) are also considered useful in the treatment of diseases relating to abnormal platelet function (e.g. occlusive vascular diseases).

The compounds of formula (I) are also considered useful for the preparation of a drug with diuretic action.

The compounds of formula (I) are also considered useful in the treatment of impotence or erectile dysfunction.

The compounds of formula (I) are also considered useful in the treatment of bone disease characterised by abnormal bone metabolism or resorbtion such as osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

The compounds of formula (I) are also considered useful for attenuating the hemodynamic side effects of non-steroidal anti-inflammatory drugs (NSAID's) and cyclooxygenase-2 (COX-2) inhibitors.

The compounds of formula (I) are also considered useful in the treatment of cardiovascular diseases such as hypertension or myocardiac ischemia; functional or organic venous insufficiency; varicose therapy; haemorrhoids; and shock states associated with a marked drop in arterial pressure (e.g. septic shock).

The compounds of formula (I) are also considered useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

The compounds of formula (I) are also considered useful in the treatment of tinnitus.

The compounds of formula (I) are also considered useful in preventing or reducing dependence on, or preventing or reducing tolerance or reverse tolerance to, a dependence-inducing agent. Examples of dependence inducing agents include opioids (e.g. morphine), CNS depressants (e.g. ethanol), psychostimulants (e.g. cocaine) and nicotine.

The compounds of formula (I) are also considered useful in the treatment of complications of Type 1 diabetes (e.g. diabetic microangiopathy, diabetic retinopathy, diabetic nephropathy, macular degeneration, glaucoma), nephrotic syndrome, aplastic anaemia, uveitis, Kawasaki disease and sarcoidosis.

The compounds of formula (I) are also considered useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

The compounds of formula (I) are also considered useful in the treatment of overactive bladder and urge incontenance.

The compounds of formula (I) are also considered useful in the treatment of lower urinary tract symptoms associated with benign prostatic hyperplasia.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof adapted for use in human or veterinary medicine.

The compounds of formula (I) and their pharmaceutically acceptable derivatives may be formulated for administration in any suitable manner. They may, for example, be formulated for administration by inhalation, or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative.

Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as diclofenac, indomethacin, nabumetone or ibuprofen; leukotriene receptor antagonists; DMARD's such as methotrexate; adenosine A1 receptor agonists; sodium channel blockers, such as lamotrigine; NMDA receptor modulators, such as glycine receptor antagonists; ligands for the α₂δ-subunit of voltage gated calcium channels, such as gabapentin and pregabalin; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; EP₄ receptor ligands; EP₂ receptor ligands; EP₃ receptor ligands; EP₄ agonists and EP₂ agonists; EP₄ antagonists; EP₂ antagonists and EP₃ antagonists; cannabanoid receptor ligands; bradykinin receptor ligands and vanilloid receptor ligand. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO 99/12930, WO 00/26216, WO 00/52008, WO 00/38311, WO 01/58881 and WO 02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A proposed daily dosage of compounds of formula (I) or their pharmaceutically acceptable derivatives for the treatment of man is from 0.01 to 30 mg/kg body weight per day and more particularly 0.1 to 10 mg/kg body weight per day, calculated as the free base, which may be administered as a single or divided dose, for example one to four times per day. The dose range for adult human beings is generally from 8 to 2000 mg/day, such as from 20 to 1000 mg/day, preferably 35 to 200 mg/day, calculated as the free base.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

No unacceptable toxicological effects are expected with compounds of the invention when administered in accordance with the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

### Abbreviations

Bn (benzyl), Bu, Pr, Me, Et (butyl, propyl, methyl ethyl), DMSO (dimethyl sulfoxide), DME (ethylene glycol dimethyl ether), EtOH (ethanol), LCMS (Liquid chromatography/Mass spectroscopy), MDAP/MDP (Mass Directed Purification), MeOH (methanol), NMR (Nuclear Magnetic Resonance (spectrum)), Ph (phenyl), SPE (Solid Phase Extraction), THF (tetrahydrofuran), s, d, t, q, m, br (singlet, doublet, triplet, quartet, multiplet, broad).

LCMS

Column: 3.3 cm×4.6 mm ID, 3 um ABZ+PLUS
Flow Rate: 3 ml/min
Injection Volume: 5 μl
Temp: RT
UV Detection Range: 215 to 330 nm
Solvents:
   A: 0.1% Formic Acid+10 mMolar Ammonium Acetate.

B: 95% Acetonitrile+0.05% Formic Acid

| Gradient: | Time | A % | B % |
|---|---|---|---|
| | 0.00 | 100 | 0 |
| | 0.70 | 100 | 0 |
| | 4.20 | 0 | 100 |
| | 5.30 | 0 | 100 |
| | 5.50 | 100 | 0 |

Mass Directed Autopreparation
Hardware:
Waters 600 gradient pump
Waters 2767 inject/collector
Waters Reagent Manager
Micromass ZMD mass spectrometer
Gilson Aspec—waste collector
Gilson 115 post-fraction UV detector
   Software:
Micromass Masslynx version 4.0
   Column
The column used is typically a Supelco LCABZ++ column whose dimensions are 20 mm internal diameter by 100 mm in length. The stationary phase particle size is 5 μm.
   Solvents:
A: Aqueous solvent=Water+0.1% Formic Acid
B: Organic solvent=MeCN: Water 95:5+0.05% Formic Acid
Make up solvent=MeOH: Water 80:20+50 mMol Ammonium Acetate
Needle rinse solvent=MeOH: Water: DMSO 80:10:10

The method used depends on the analytical retention time of the compound of interest. 15-minute runtime, which comprises a 10-minute gradient followed by a 5-minute column flush and re-equilibration step.
MDP 1.5-2.2=0-30% B
MDP 2.0-2.8=5-30% B
MDP 2.5-3.0=15-55% B
MDP 2.8-4.0=30-80% B
MDP 3.8-5.5=50-90% B
   Flow Rate:
flow rate 20 ml/min.

### Intermediate Synthesis

### 5-Bromo-2′-iodo-2-(methyloxy)biphenyl

A mixture of 1,2-diiodobenzene (35 g, 106 mmol), 5-bromo-2-methyloxyphenylboronic acid (20 g, 85 mmol), tetrakis(triphenylphosphine) palladium(0) (1.0 g) and potassium carbonate (11.0 g) in 1,2 dimethoxyethane (120 ml) was refluxed under nitrogen for 80 hours. The reaction mixture was filtered through celite and flash chromatographed, eluting with dichloromethane/iso-hexane (1/4) to give the title compound. (19.0 g). LC/MS Rt=3.9 min.

### [5′-Bromo-2′-(methyloxy)-2-biphenylyl]boronic acid

5-Bromo-2′-iodo-2-(methyloxy)biphenyl (1.6 g, 4 mmol) in dry tetrahydrofuran (30 ml) was cooled to −40° C. under nitrogen and treated dropwise with 2 molar isopropylmagnesium chloride in tetrahydrofuran (5 ml, 10 mmol). The reaction mixture was stirred under nitrogen at −40° C. for 1 hour, cooled to −75° C. and slowly treated with triisopropyl borate (3 ml, 13 mmol). The reaction mixture was then allowed to reach room temperature and stirred overnight. The reaction mixture was quenched with 2N hydrochloric acid (30 ml), stirred vigorously for 15 min then diluted with diethyl ether (30 ml). The organic layer was separated, dried over magnesium sulphate, filtered and evaporated to an oil which was purified by flash chromatography first with diethyl ether/isohexane (30/100), then with diethyl ether/methanol (100/2) to give the title compound (0.77 g). LC/MS Rt=2.9 min.

### Ethyl 6-[5′-bromo-2′-(methyloxy)-2-biphenylyl]-2-pyridinecarboxyiate

A mixture of [5′-bromo-2′-(methyloxy)-2-biphenylyl]boronic acid (11.5 g, 37 mmol), ethyl-6-bromopicolinate (11.0 g, 47 mmol), tetrakis(triphenylphosphine) palladium(0) (1.5 g) and potassium carbonate (20 g) in 1,2-dimethoxyethane (100 ml) was refluxed with vigorous stirring under nitrogen for 72 hours. The reaction mixture was then filtered through celite, washed with dichloromethane and evaporated to an oil which was flash chromatographed eluting with diethyl ether/iso-hexane (1/9) to give the title compound (9.8 g).

LC/MS [M+H] 414.3, Rt=3.4 min.

### 6-(5′-Bromo-2′-hydroxy-2-biphenylyl)-2-pyridinecarboxylic acid

Ethyl 6-[5′-bromo-2′-(methyloxy)-2-biphenylyl]-2-pyridinecarboxylate (9.8 g, 23 mmol) in dry dichloromethane (200 ml) was cooled to −75° C. with vigorous stirring under nitrogen and treated with boron tribromide (18 mls, 190 mmol) dropwise. The reaction mixture was then stirred at −75° C. for 2 hours and the temperature was allowed to rise to 0° C. The mixture was quenched with ice/water (400 ml). After stirring for 1 hour, the organic layer was separated, dried over magnesium sulphate, filtered, evaporated and flash chromatographed with dichloromethane then with dichloromethane/methanol (95/5) to give the title compound (5.4 g). LC/MS [M+H] 372.3, Rt=3.4 mins.

### Methyl 6-(5′-Bromo-2′-hydroxy-2-biphenylyl)-2-pyridinecarboxylate

6-(5′-Bromo-2′-hydroxy-2-biphenylyl)-2-pyridinecarboxylic acid (5.0 g, 13 mmol) in dry methanol (100 ml) was treated with concentrated sulphuric acid (0.5 ml) and heated to reflux for 6 hours. The reaction mixture was evaporated to 10 mls, cooled to 0° C. and neutralised with ammonia. The reaction mixture was then partitioned between water (50 ml) and ethyl acetate (50 ml). The organic layer was separated, dried over magnesium sulphate, filtered and evaporated to give the title compound (4.8 g).

LC/MS [M+H] 386.3, 387.3, Rt=3.1 min.

### Methyl 6-{5′-bromo-2′-[(2-methyl-2-propen-1-yl)oxy]-2-biphenylyl}-2-pyridinecarboxylate

A mixture of methyl 6-(5′-bromo-2′-hydroxy-2-biphenylyl)-2-pyridinecarboxylate (88 mg, 0.23 mmol), potassium carbonate (81 mg, 0.58 mmol) and 3-bromo-2-methylpropene (26 μL, 0.26 mmol) was heated in N,N′-dimethylformamide (3 ml) at 90° C. for 14 hours. After cooling the reaction mixture was evaporated and the residue was flash chromatographed using 15% ethyl acetate in iso-hexane as eluent to yield the title compound as a clear liquid (98 mg). LC/MS: Rt=3.95 min. [MH⁺]=438, 441.

The following compounds were prepared from appropriate intermediates in a similar manner to methyl 6-{5′-bromo-2′-[(2-methyl-2-propen-1-yl)oxy]-2-biphenylyl}-2-pyridinecarboxylate:

| Compound name | LC/MS |
|---|---|
| Methyl 6-[5′-bromo-2′-(2-propyn-1- | [MH⁺] = 424, 425, |
| yloxy)-2-biphenylyl]-2- | Rt = 3.59 min |
| pyridinecarboxylate | |
| Methyl 6-{5′-bromo-2′-[(3-phenyl-2- | [MH⁺] = 500, 501, |
| propyn-1-yl)oxy]-2-biphenylyl}-2- | Rt = 4.11 min |
| pyridinecarboxylate | |
| Methyl 6-[5′-bromo-2′-(2-butyn-1-yloxy)- | [MH⁺] = 438, 439, |
| 2-biphenylyl]-2-pyridinecarboxylate | Rt = 3.72 min |
| Methyl 6-[5′-bromo-2′-(2-pentyn-1- | [MH⁺] = 452, 453 |
| yloxy)-2-biphenylyl]-2- | Rt = 3.94 min |
| pyridinecarboxylate | |

### Methyl 6-{5′-bromo-2′-[(2,2-dimethylpropyl)oxy]-2-biphenylyl}-2-pyridinecarboxylate

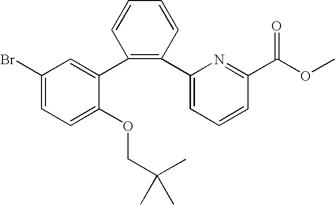

A mixture of methyl 6-(5′-bromo-2′-hydroxy-2-biphenylyl)-2-pyridinecarboxylate (88 mg, 0.23 mmol), potassium carbonate (81 mg, 0.58 mmol), and 1-bromo-2,2-dimethylpropane (32 μL, 0.26 mmol) in N,N′-dimethylformamide (2 ml) was reacted in a microwave at 180° C. for 2.5 hrs. The mixture was then cooled to room temperature and evaporated. The residue was flash chromatographed using 15-20% ethyl acetate in iso-hexane as eluent to yield the title compound as a clear liquid (16 mg). LC/MS: Rt=4.26 min. [MH⁺]=456, 457.

The following compound was prepared from appropriate intermediates in a similar manner to methyl 6-{5′-bromo-2′-[(2,2-dimethylpropyl)oxy]-2-biphenylyl}-2-pyridinecarboxylate:

| Compound name | LC/MS |
|---|---|
| Methyl 6-{5′-bromo-2′-[(2,2,2- | [MH⁺] = 466, 469 Rt = 3.77 |
| trifluoroethyl)oxy]-2-biphenylyl}-2- | min |
| pyridinecarboxylate | |

### Methyl 6-{5′-bromo-2′-[(2-methylbutyl)oxy]-2-biphenylyl}-2-pyridinecarboxylate

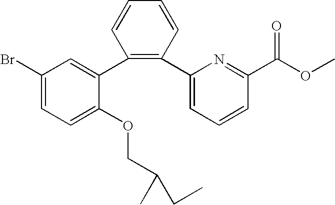

A mixture of methyl 6-(5′-bromo-2′-hydroxy-2-biphenylyl)-2-pyridinecarboxylate (90 mg, 0.23 mmol), 2-methyl-1-butanol (23 μL, 0.21 mmol), triphenylphosphine (61 mg, 0.23 mmol) and diisopropyl azodicarboxylate (46 μL, 0.23 mmol) in tetrahydrofuran (4 ml) was stirred at room temperature overnight. The mixture was then evaporated and the residue was flash chromatographed using 15% ethyl acetate in iso-hexane as eluent to yield the title compound (60 mg) as a clear oil. LC/MS: Rt=4.27 min. [MH⁺]=456, 457.

The following compound was prepared from appropriate intermediates in a similar manner to methyl 6-{5′-bromo-2′-[(2-methylbutyl)oxy]-2-biphenylyl}-2-pyridinecarboxylate:

| Compound name | LC/MS |
|---|---|
| Methyl 6-{5′-bromo-2′-[(3,3,3- | [MH⁺] = 482, 483, |
| trifluoropropyl)oxy]-2-biphenylyl}-2- | Rt = 3.92 min |
| pyridinecarboxylate | |

### Example 1

### Sodium 6-{5′-bromo-2′-[(2-methyl-2-propen-1-yl)oxy]-2-biphenylyl}-2-pyridinecarboxylate

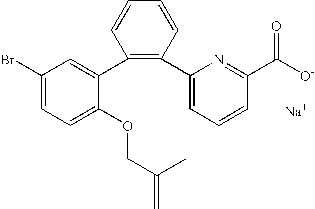

Ethyl 6-{5′-bromo-2′-[(2-methyl-2-propen-1-yl)oxy]-2-biphenylyl}-2-pyridinecarboxylate (˜100 mg, 0.22 mmol) was suspended in ethanol (3 ml), 1M sodium hydroxide (1 ml) added and the reaction stirred at room temperature for 2 hours. The mixture was evaporated to dryness, diluted with water and extracted with ethyl acetate (×3), the organic layer dried (MgSO₄) and evaporated to give the title compound (95 mg).

LC/MS: Rt=3.52 [MH⁻]=422, 424.

The following compounds were prepared from appropriate intermediates in a similar manner to sodium 6-{5′-bromo-2′-[(2-methyl-2-propen-1-yl)oxy]-2-biphenylyl}-2-pyridinecarboxylate:

| EXAM- | | |
|---|---|---|
| PLE | COMPOUND NAME | LC/MS |
| 2 | Sodium 6-{5′-bromo-2′- | [MH⁺] = 442, 443 [MH⁻] = 440, |
| | [(2-(R,S)- | 441, Rt = 4.21 min |
| | methylbutyl)oxy]-2- | |
| | biphenylyl}-2- | |
| | pyridinecarboxylate | |
| 3 | Sodium 6-{5′-bromo-2′- | [MH⁺] = 442, 443 [MH⁻] = 440, |
| | [(2,2-dimethylpropyl)oxy]- | 441, Rt = 4.26 min |
| | 2-biphenylyl}-2- | |
| | pyridinecarboxylate | |
| 4 | Sodium 6-[5′-bromo-2′-(2- | [MH⁺] = 410, 411, Rt = 3.42 min |
| | propyn-1-yloxy)-2- | |
| | biphenylyl]-2- | |
| | pyridinecarboxylate | |
| 5 | Sodium 6-{5′-bromo-2′- | [MH⁺] = 424, 425, Rt = 3.60 min |
| | [(3-phenyl-2-propyn-1- | |
| | yl)oxy]-2-biphenylyl}-2- | |
| | pyridinecarboxylate | |
| 6 | Sodium 6-[5′-bromo-2′-(2- | [MH⁺] = 454, 455, Rt = 3.59 min |
| | butyn-1-yloxy)-2- | |
| | biphenylyl]-2- | |
| | pyridinecarboxylate | |
| 7 | Sodium 6-{5′-bromo-2′- | [MH⁺] = 466, 469, Rt = 3.74 min |
| | [(2,2,2-trifluoroethyl)oxy]- | |
| | 2-biphenylyl}-2- | |
| | pyridinecarboxylate | |
| 8 | Sodium 6-{5′-bromo-2′- | [MH⁺] = 438, 439, Rt = 3.81 min |
| | [(3,3,3- | |
| | trifluoropropyl)oxy]-2- | |
| | biphenylyl}-2- | |
| | pyridinecarboxylate | |
| 9 | Sodium 6-[5′-bromo-2′-(2- | [MH⁺] = 482, 483, Rt = 3.92 min |
| | pentyn-1-yloxy)-2- | |
| | biphenylyl]-2- | |
| | pyridinecarboxylate | |

### Example 10

### Sodium 6-{5′-bromo-2′-[(phenylmethyl)oxy]-2-biphenylyl}-2-pyridinecarboxylate

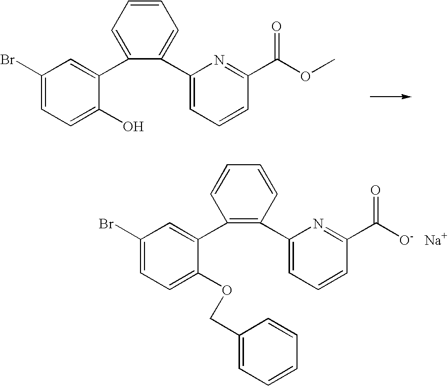

A mixture of methyl 6-(5′-bromo-2′-hydroxy-2-biphenylyl)-2-pyridinecarboxylate (200 mg, 0.52 mmol), benzyl bromide (95 mg, 0.55 mmol) and potassium carbonate (200 mg) in 2-butanone (4 ml) was heated to reflux overnight under nitrogen. The reaction mixture was cooled and filtered through celite which was washed with ethyl acetate (10 mls). The filtrate was diluted with water (20 ml) and further ethyl acetate (10 ml). The organic layer was separated and dried over magnesium sulfate and evaporated to an oil which was dissolved in methanol (5 ml) and treated with 2N sodium hydroxide (2 ml). The mixture was stirred at 70° C. for 15 mins then evaporated. Water (20 ml) was added and the mixture extracted with ethyl acetate (2×40 ml). After drying over sodium sulphate, the ethyl acetate extract was evaporated to give the title compound (200 mg).

LC/MS [M+H] 462.3, 463.3, Rt=3.55 min.

The following examples were prepared from the appropriate intermediates in a similar manner to sodium 6-{5′-bromo-2′-[(phenylmethyl)oxy]-2-biphenylyl}-2-pyridinecarboxylate:

| EXAM- | | |
|---|---|---|
| PLE | COMPOUND NAME | LC/MS |
| 11 | Sodium 6-(5′-bromo-2′-{[(4-chloro-2- | [M + H] 514.2, 516.3 |
| | fluorophenyl)methyl]oxy}-2- | Rt = 3.76 min |
| | biphenylyl)-2-pyridinecarboxylate | |
| 12 | Sodium 6-(5′-bromo-2′-{[(2,4,5- | [M + H] 516.3, 517.3 |
| | trifluorophenyl)methyl]oxy}-2- | Rt = 3.80 min |
| | biphenylyl)-2-pyridinecarboxylate | |
| 13 | Sodium 6-(5′-bromo-2′-{[(2-chloro-4- | [M + H] 514.2, 516.3 |
| | fluorophenyl)methyl]oxy}-2- | Rt = 3.90 min |
| | biphenylyl)-2-pyridinecarboxylate | |
| 14 | Sodium 6-(5′-bromo-2′-{[(2,3,6- | [M + H] 517.3, 517.2 |
| | trifluorophenyl)methyl]oxy}-2- | Rt-3.89 min |
| | biphenylyl)-2-pyridinecarboxylate | |
| 15 | Sodium 6-(5′-bromo-2′-{[(4- | [M + H] 496.3, 498.3 |
| | chlorophenyl)methyl]oxy}-2- | Rt = 3.88 min |
| | biphenylyl)-2-pyridinecarboxylate | |
| 16 | Sodium 6-(5′-bromo-2′-{[(2,6- | [M + H] 498.3, 499.3 |
| | difluorophenyl)methyl]oxy}-2- | Rt = 3.76 min |
| | biphenylyl)-2-pyridinecarboxylate | |
| 17 | Sodium 6-(5′-bromo-2′-[(tetrahydro- | [M + H] 456.3, 457.3 |
| | 2-(R,S)-furanylmethyl)oxy]-2- | Rt = 3.24 min |
| | biphenylyl-2-pyridinecarboxylate | |
| 18 | Sodium 6-[5′-bromo-2′-[{[4- | [M + H] 530.3, 531.3 |
| | (trifluoromethyl)phenyl]methyl}oxy)-2- | Rt = 4.08 min |
| | biphenylyl]-2-pyridinecarboxylate | |
| 19 | Sodium 6-(5′-bromo-2′-{[(2,4- | [M + H] 498.2, 499.3 |
| | difluorophenyl)methyl]oxy}-2- | Rt = 3.76 min |
| | biphenylyl)-2-pyridinecarboxylate | |
| 20 | Sodium 6-(5′-bromo-2′-{[(2,4,6- | [M + H] 516.1, 517.1 |
| | trifluorophenyl)methyl]oxy}-2- | Rt = 3.88 min |
| | biphenylyl)-2-pyridinecarboxylate | |
| 21 | Sodium 6-(5′-bromo-2′-{[(4- | [M + H] 480.2, 481.3 |
| | fluorophenyl)methyl]oxy}-2- | Rt = 3.56 min |
| | biphenylyl-2-pridinecarboxylate | |

### Example 22

### Sodium 6-{5′-bromo-2′-[(cyclohexylmethyl)oxy]-2-biphenylyl}-2-pyridinecarboxylate

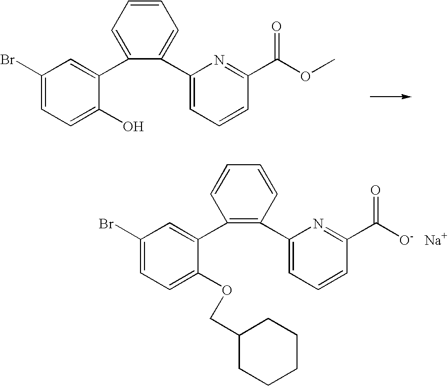

A mixture of methyl 6-(5′-bromo-2′-hydroxy-2-biphenylyl)-2-pyridinecarboxylate (200 mg, 0.52 mmol), (bromomethyl)cyclohexane (260 mg, 1.5 mmol) and potassium cabonate (200 mg) in dimethylformamide (4 ml) was heated to reflux for 3 hours under nitrogen. The reaction mixture was then filtered through celite which was washed with ethyl acetate (10 mls) and the filtrate evaporated to an oil which was flash chromatographed eluting with diethyl ether/isohexane (1/5). The product was dissolved in methanol (10 ml), treated with 2N sodium hydroxide (2 ml) and heated at 70° C. for 15 min. The solution was evaporated and partitioned between water and ethyl acetate. After drying with anhydrous sodium sulphate the ethyl acetate solution was evaporated to give the title compound (150 mg).

LC/MS [M+H] 468.3, 469.4, Rt=3.91 min.

The following Examples were prepared in a similar manner to sodium 6-{5′-bromo-2′-[(cyclohexylmethyl)oxy]-2-biphenylyl}-2-pyridinecarboxylate (Example 22)

| EXAM- | | |
|---|---|---|
| PLE | COMPOUND NAME | LC/MS |
| 23 | Sodium 6-{5′-bromo-2′-[(2- | [M + H] 428.3, 429.3 |
| | methylpropyl)oxy]-2-biphenylyl}-2- | Rt = 3.82 min |
| | pyridinecarboxylate | |
| 24 | Sodium 6-{5′-bromo-2′- | [M + H] 440.4, 441.3 |
| | [(cyclobutylmethyl)oxy]-2-biphenylyl}- | Rt = 3.84 min |
| | 2-pyridinecarboxylate | |
| 25 | Sodium 6-{5′-bromo-2′- | [M + H] 426.3, 427.4 |
| | [(cyclopropylmethyl)oxy]-2- | Rt = 3.84 min |
| | biphenylyl}-2-pyridinecarboxylate | |
| 26 | Sodium 6-{5′-bromo-2′-[(3- | [M + H] 442.3, 443.3 |
| | methylbutyl)oxy]-2-biphenylyl}-2- | Rt = 4.26 min |
| | pyridinecarboxylate | |
| 27 | Sodium 6-{5′-bromo-2′-[(2- | [M + H] 456.3, 457.3 |
| | ethylbutyl)oxy]-2-biphenylyl}-2- | Rt = 4.48 min |
| | pyridinecarboxylate | |
| 28 | Sodium 6-[5′-bromo-2′-(propyloxy)-2- | [M + H] 414.3, 415.3 |
| | biphenylyl]-2-pyridinecarboxylate | Rt = 3.94 min |
| 29 | Sodium 6-{5′-bromo-2′-[(1- | [M + H] 414.3, 415.3 |
| | methylethyl)oxy]-2-biphenylyl}-2- | Rt = 4.04 min |
| | pyridinecarboxylate | |

### Example 30

### Sodium 6-{5′-bromo-2′-[(cyclopentylmethyl)oxy]-2-biphenylyl}-2-pyridinecarboxylate

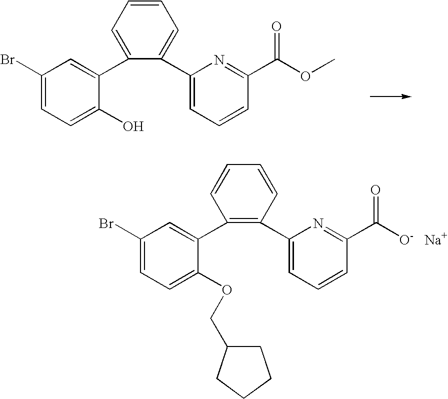

Methyl 6-(5′-bromo-2′-hydroxy-2-biphenylyl)-2-pyridinecarboxylate (200 mg, 0.52 mmol) was dissolved in tetrahydofuran (3 ml) and treated with triphenylphosphine (290 mg, 1.1 mmol), di-tert-butyl azodicarboxylate (190 mg, 0.8 mmol) and cyclopentylmethanol (160 mg, 1.6 mmol). The reaction mixture was stirred under nitrogen overnight at room temperature. The reaction mixture was diluted with ethyl acetate (30 ml) and water (20 ml) added. The organic layer was dried over magnesium sulphate and evaporated. The residue was flash chromatographed eluting with diethyl ether/isohexane (1/5). The product was dissolved in methanol (10 ml), treated with 2N sodium hydroxide (2 ml) and heated at 70° C. for 15 min. The solution was evaporated and partitioned between water and ethyl acetate. After drying with anhydrous sodium sulphate the ethyl acetate solution was evaporated to give the title compound (80 mg). LC/MS [M+H] 454.4, 455.3, Rt=4.31 min.

### Example 31

### Sodium 6-{5′-bromo-2′-[(tetrahydro-3-(R,S)-furanylmethyl)oxy]-2-biphenylyl}-2-pyridinecarboxylate

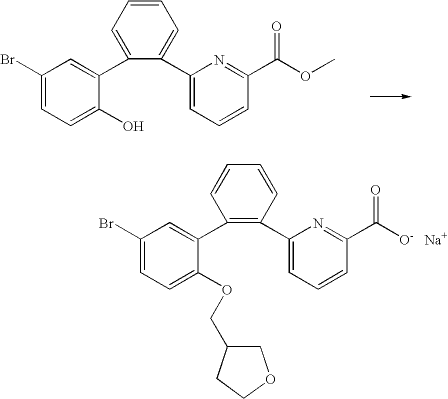

The title compound was prepared in a similar manner to sodium 6-{5′-bromo-2′-[(cyclopentylmethyl)oxy]-2-biphenylyl}-2-pyridinecarboxylate.

LC/MS [M+H] 470.4, 471.4, Rt=3.65 min.

### Example 32

### Sodium 6-{5′-bromo-2′-[(tetrahydro-2H-pyran-4-ylmethyl)oxy]-2-biphenylyl}-2-pyridinecarboxylate

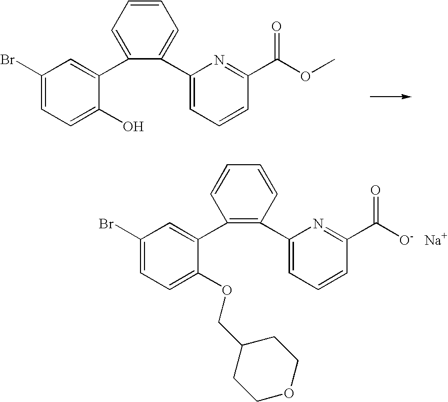

A mixture of methyl 6-(5′-bromo-2′-hydroxy-2-biphenylyl)-2-pyridinecarboxylate (200 mg, 0.52 mmol), tetrahydro-2H-pyran-4-ylmethyl-4-methylbenzene sulphonate (430 mg, 1.5 mmol) and potassium cabonate (200 mg) in dimethylformamide (4 ml) was heated to reflux for 3 hours under nitrogen. The reaction mixture was then filtered through celite, washed with ethyl acetate (10 mls) and evaporated to an oil which was flash chromatographed eluting with diethyl ether/isohexane (1/5). The product was dissolved in methanol (10 ml), treated with 2N sodium hydroxide (2 ml) and heated at 70° C. for 15 min. The solution was evaporated and partitioned between water and ethyl acetate. After drying with anhydrous sodium sulphate the ethyl acetate solution was evaporated to give the title compound (130 mg). LC/MS [M+H] 470.3, 471.4, Rt=3.65 min.

### Intermediate Synthesis

### 2′-Bromo-5-chloro-2-biphenylyl phenylmethyl ether

A mixture of 4-chloro-2-iodophenyl phenylmethyl ether (34.0 g, 99 mmol), 2-bromophenyl boronic acid (29.7 g, 149 mmol), potassium carbonate (54.5 g, 396 mmol), and tetrakis(triphenylphosphine) palladium(0) (4 g, 3.46 mmol) was refluxed under nitrogen in toluene/ethanol (1/1, 500 ml) for 3 hours. This was diluted with diethyl ether (1 l) and washed twice with water (500 ml), once with brine (250 ml), dried over sodium sulfate, filtered and evaporated to an oil which was purified by flash chromatography eluting with iso-hexane/dichloromethane (19/1) to give the title compound (18.52 g), LC/MS Rt=4.21 min.

### {5′-Chloro-2′-[(phenylmethyl)oxy]-2-biphenylyl}boronic acid

2′-Bromo-5-chloro-2-biphenylyl phenylmethyl ether (18.5 g, 50 mmol) was stirred in tetrahydrofuran (290 ml), cooled to −75° C. and treated dropwise with 1.6 molar n-BuLi in hexanes (31.25 ml, 50 mmol). The reaction was stirred for 30 mins and then slowly treated with triisopropyl borate (24 ml, 200 mmol). The reaction was allowed to come to room temperature over 1 hour and then heated to 50° C. for another 30 mins. 2N hydrochloric acid (250 ml) was added and the reaction diluted with diethyl ether (500 ml). The aqueous layer was separated and the organic layer washed with water (250 ml) and brine (250 ml) and dried over sodium sulfate. The solution was evaporated to a yellow oil and flash chromatographed with first dichloromethane and then ethyl acetate to give the title compound (11.3 g). LC/MS Rt=3.43 min.

### Ethyl 6-{5′-chloro-2′-[(Phenylmethyl)oxy]-2-biphenylyl}-2-pyridinecarboxylate

A mixture of {5′-chloro-2′-[(phenylmethyl)oxy]-2-biphenylyl}boronic acid (11.3 g, 33.4 mmol), ethyl 6-bromo-2-pyridinecarboxylate (7.3 g, 31.7 mmol), potassium carbonate (23 g, 167 mmol) and tetrakis(triphenylphosphine)palladium(0) (1.93 g, 1.67 mmol) was stirred at reflux in ethanol/toluene (200 ml, 1/1) for 1 hour. The reaction was acidified to pH1 with 2N hydrochloric acid. The aqueous layer was discarded and the organic layer washed with water, brine, dried over sodium sulphate and then evaporated to a yellow oil. This was flash chromatographed with ethyl acetate/iso-hexane (1/9) to give the title compound (11 g). LC/MS [M+H] 444, 446, Rt=4.05 min

### Ethyl 6-(5′-chloro-2′-hydroxy-2-biphenylyl)-2-pyridinecarboxylate

Ethyl 6-{5′-chloro-2′-[(phenylmethyl)oxy]-2-biphenylyl}-2-pyridinecarboxylate (6.5 g, 14.6 mmol) was dissolved in acetic acid (60 ml). 48% Hydrogen bromide in acetic acid (70 ml) was added and the reaction stirred for 1 hour. The mixture was diluted with diethyl ether (500 ml) and potassium carbonate added to pH12. The organic layer was washed with water, brine and evaporated to an oil which was purified by flash chromatography, eluting with iso-hexane/ethyl acetate (3/1) to give the title compound (2.5 g). LC/MS [M+H] 354, 356, Rt=3.43 min.

### Ethyl 6-(5′-chloro-2′-{[(2-fluorophenyl)methyl]oxy}-2-biphenylyl)-2-pyridinecarboxylate

A mixture of ethyl 6-(5′-chloro-2′-hydroxy-2-biphenylyl)-2-pyridinecarboxylate (0.15 g, 0.42 mmol), 2-fluorobenzyl bromide (0.08 ml, 0.5 mmol) and potassium carbonate (0.09 g, 0.63 mmol) were refluxed in acetone for 18 hours, filtered and evaporated to an oil which was flash chromatographed with iso-hexane/ethyl acetate (9/1) to give the title compound (0.18 g). LC/MS [M+H] 462, 464, Rt=4.07 min.

The following compounds were prepared in a simliar manner to ethyl 6-(5′-chloro-2′-{[(2-fluorophenyl)methyl]oxy}-2-biphenylyl)-2-pyridinecarboxylate:

| Compound name | LC/MS |
|---|---|
| Ethyl 6-[5′-chloro-2′-({[2-fluoro-4- | [M + H] 530, 462, Rt = 4.31 |
| (trifluoromethyl)phenyl]methyl}oxy)-2- | min |
| biphenylyl]-2-pyridinecarboxylate | |
| Ethyl 6-(5′-chloro-2′-{[(2-chloro-6- | [M + H] 496, 498, Rt = 4.13 |
| fluorophenyl)methyl]oxy}-2- | min |
| biphenylyl)-2-pyridinecarboxylate | |
| Ethyl 6-(5′-chloro-2′-{[(2,4,6- | [M + H] 498, 500, Rt = 4.06 |
| trifluorophenyl)methyl]oxy}-2- | min |
| biphenylyl)-2-pyridinecarboxylate | |
| Ethyl 6-(5′-chloro-2′-{[(2-chloro-4- | [M + H] 496, 498, Rt = 4.26 |
| fluorophenyl)methyl]oxy}-2- | min |
| biphenylyl)-2-pyridinecarboxylate | |
| Ethyl 6-(2′-{[(4-bromo-2- | [M + H] 540, 542, Rt = 4.29 |
| fluorophenyl)methyl]oxy}-5′-chloro-2- | min |
| biphenylyl)-2-pyridinecarboxylate | |
| Ethyl 6-(5′-chloro-2′-{[(4-chloro-2- | [M + H] 496, 498, Rt = 4.25 |
| fluorophenyl)methyl]oxy}-2- | min |
| biphenylyl)-2-pyridinecarboxylate | |
| Ethyl 6-(5′-chloro-2′-{[(2,6- | [M + H] 480, 482, Rt = 3.99 |
| difluorophenyl)methyl]oxy}-2- | min |
| biphenylyl)-2-pyridinecarboxylate | |
| Ethyl 6-(5′-chloro-2′-{[(2- | [M + H] 478, 480, Rt = 4.21 |
| chlorophenyl)methyl]oxy}-2- | min |
| biphenylyl)-2-pyridinecarboxylate | |
| Ethyl 6-(5′-chloro-2′-{[(3,4,5- | [M + H] 498, 500, Rt = 4.18 |
| trifluorophenyl)methyl]oxy}-2- | min |
| biphenylyl)-2-pyridinecarboxylate | |

### Ethyl 6-{5′-chloro-2′-[(2-methylpropyl)oxy]-2-biphenylyl}-2-pyridinecarboxylate

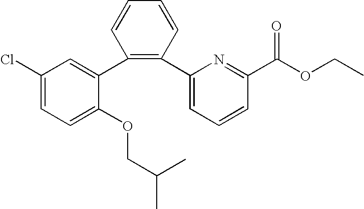

A mixture of ethyl 6-(5′-chloro-2′-hydroxy-2-biphenylyl)-2-pyridinecarboxylate (0.15 g, 0.42 mmol), 1-bromo-2-methylpropane (0.054 ml, 0.5 mmol) and potassium carbonate (0.09 g, 0.63 mmol) were stirred in dimethylformamide for 20 hours, filtered and evaporated to an oil which was flash chromatographed with iso-hexane/ethyl acetate (9/1) to give the title compound (0.15 g). LC/MS [M+H] 410, 412, Rt=4.15 min.

The following compounds were prepared in a simliar manner to ethyl 6-{5′-chloro-2′-[(2-methylpropyl)oxy]-2-biphenylyl}-2-pyridinecarboxylate:

| Compound name | LC/MS |
|---|---|
| Ethyl 6-{5′-chloro-2′- | LC/MS[M + H] 422, 424, |
| [(cyclobutylmethyl)oxy]-2-biphenylyl}- | Rt = 4.18 min |
| 2-pyridinecarboxylate | |
| Ethyl 6-{5′-chloro-2′- | LC/MS[M + H] 408, 410, |
| [(cyclopropylmethyl)oxy]-2- | Rt = 3.93 min |
| biphenylyl}-2-pyridinecarboxylate | |
| Ethyl 6-{5′-chloro-2′-[(tetrahydro-2- | LC/MS[M + H] 438, 440, |
| furanylmethyl)oxy]-2-biphenylyl}-2- | Rt = 3.78 min |
| pyridinecarboxylate | |
| Ethyl 6-{5′-chloro-2′- | LC/MS[M + H] 450, 452, |
| [(cyclohexylmethyl)oxy]-2-biphenylyl}- | Rt = 4.45 min |
| 2-pyridinecarboxylate | |

### Ethyl 6-{5′-chloro-2′-[(cyclopentylmethyl)oxy]-2-biphenylyl}-2-pyridinecarboxylate

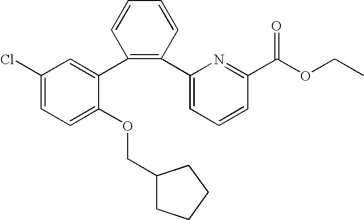

A mixture of ethyl 6-(5′-chloro-2′-hydroxy-2-biphenylyl)-2-pyridinecarboxylate (0.15 g, 0.42 mmol), dibutyl azodicarboxylate (1.3 mmol, 0.31 g), cyclopentylmethyl alcohol (1.3 mmol, 0.057 ml) and triphenyl phosphine (1.68 mmol, 0.44 g) were stirred under nitrogen in anhydrous tetrahydrofuran (5 ml) for 18 hours. This was then evaporated to an oil and flash chromatographed with iso-hexane/ethyl acetate (9/1) to give the title compound (0.164 g) LC/MS [M+H] 436 and 438, Rt=4.34 min.

### Example 33

### Sodium 6-{5′-chloro-2′-[(2-methylpropyl)oxy]-2-biphenylyl}-2-pyridinecarboxylate

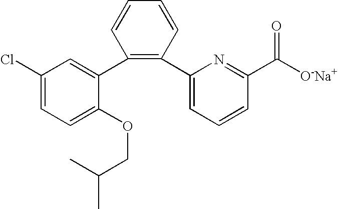

Ethyl 6-{5′-chloro-2′-[(2-methylpropyl)oxy]-2-biphenylyl}-2-pyridinecarboxylate (0.14 g, 0.35 mmol) was suspended in ethanol (10 ml), 2N sodium hydroxide added and the reaction stirred at 70° C. for 15 mins. The mixture was evaporated to dryness, partitioned between ethyl acetate (20 ml) and water (10 ml) and the organic layer dried over sodium sulfate, filtered and the filtrate evaporated to give the title compound (0.137 g) LC/MS [M+H] 382, 384, Rt=3.91 min.

¹H NMR (DMSO) δ 0.76 (d, J=6.68 Hz, 6H), 1.75-1.85 (m, 1H), 3.57 (d, J=6.15, 2H), 6.67 (d, J=7.66 Hz, 1H), 6.89 (d, J=2.68 Hz, 1H), 7.01 (d, J=8.88 Hz, 1H), 7.24 (d, J=2.71, 1H), 7.33 (d, J=1.28 Hz, 1H) 7.36-7.50 (m, 3H), 7.67 (d, J=7.63 Hz, 1H), 7.75 (d, J=8.96 Hz, 1H).

The following Examples were prepared from the appropriate intermediate in a similar manner to sodium 6-{5′-chloro-2′-[(2-methylpropyl)oxy]-2-biphenylyl}-2-pyridinecarboxylate:

| EXAM- | | |
|---|---|---|
| PLE | COMPOUND NAME | LC/MS |
| 34 | Sodium 6-(5′-chloro-2′-{[(2- | [M + H] 434 and |
| | fluorophenyl)methyl]oxy}-2- | 436, Rt = 3.87 min |
| | biphenylyl)-2-pyridinecarboxylate | |
| 35 | Sodium 6-[5′-chloro-2′-({[2-fluoro-4- | [M + H] 502 and |
| | (trifluoromethyl)phenyl]methyl}oxy)-2- | 504, Rt = 4.10 min |
| | biphenylyl]-2-pyridinecarboxylate | |
| 36 | Sodium 6-(5′-chloro-2′-{[(2-chloro-6- | [M + H] 468 and |
| | fluorophenyl)methyl]oxy}-2- | 470, Rt = 3.93 min |
| | biphenylyl)-2-pyridinecarboxylate | |
| 37 | Sodium 6-(5′-chloro-2′-{[(2,4,6- | [M + H] 470 and |
| | trifluorophenyl)methyl]oxy}-2- | 472, Rt = 3.82 min |
| | biphenylyl)-2-pyridinecarboxylate | |
| 38 | Sodium 6-{5′-chloro-2′- | [M + H] 394 and |
| | [(cyclobutylmethyl)oxy]-2-biphenylyl}- | 396, Rt = 3.98 min |
| | 2-pyridinecarboxylate | |
| 39 | Sodium 6-{5′-chloro-2′- | [M + H] 380 and |
| | [(cyclopropylmethyl)oxy]-2- | 382, Rt = 3.68 min |
| | biphenylyl}-2-pyridinecarboxylate | |
| 40 | Sodium 6-{5′-chloro-2′-[(tetrahydro-2- | [M + H] 410 and |
| | (R,S)-furanylmethyl)oxy]-2- | 412, Rt = 3.38 min |
| | biphenylyl}-2-pyridinecarboxylate | |
| 41 | Sodium 6-{5′-chloro-2′- | [M + H] 408 and |
| | [(cyclopentylmethyl)oxy]-2- | 410, Rt = 4.14 min |
| | biphenylyl}-2-pyridinecarboxylate | |
| 42 | Sodium 6-{5′-chloro-2′- | [M + H] 422 and |
| | [(cyclohexylmethyl)oxy]-2- | 424, Rt = 4.29 min |
| | biphenylyl}-2-pyridinecarboxylate | |
| 43 | Sodium 6-(5′-chloro-2′-{[(2-chloro-4- | [M + H] 468 and |
| | fluorophenyl)methyl]oxy}-2- | 472, Rt = 4.09 min |
| | biphenylyl)-2-pyridinecarboxylate | |
| 44 | Sodium 6-(2′-{[(4-bromo-2- | [M + H] 512, 514, |
| | fluorophenyl)methyl]oxy}-5′-chloro-2- | and 516, Rt = 4.13 |
| | biphenylyl)-2-pyridinecarboxylate | min |
| 45 | Sodium 6-(5′-chloro-2′-{[(4-chloro-2- | [M + H] 468 and |
| | fluorophenyl)methyl]oxy}-2- | 470, Rt = 4.07 min |
| | biphenylyl)-2-pyridinecarboxylate | |
| 46 | Sodium 6-(5′-chloro-2′-{[(2,6- | [M + H] 452 and |
| | difluorophenyl)methyl]oxy}-2- | 454, Rt = 3.76 min |
| | biphenylyl)-2-pyridinecarboxylate | |
| 47 | Sodium 6-(5′-chloro-2′-{[(2- | [M + H] 450 and |
| | chlorophenyl)methyl]oxy}-2- | 452, Rt = 4.05 min |
| | biphenylyl)-2-pyridinecarboxylate | |
| 48 | Sodium 6-(5′-chloro-2′-{[(3,4,5- | [M + H] 470 and |
| | trifluorophenyl)methyl]oxy}-2- | 472, Rt = 3.96 min |
| | biphenylyl)-2-pyridinecarboxylate | |

### Intermediate Synthesis

### 1-Bromo-2-iodo-3-methylbenzene

A solution of sodium nitrite (1.04 g) in water (5 ml) was added dropwise to a stirred mixture of 2-bromo-6-methylaniline (2.57 g), conc. hydrochloric acid (5 ml) and water (5 ml) at 0°. The mixture was stirred for 5 minutes and a solution of potassium iodide (2.75 g) in water (10 ml) added dropwise at 0°. After stirring at ambient temperature for 21 h, the solution was extracted with dichloromethane (5×15 ml) and the combined extracts washed sequentially with water (30 ml), 5% sodium hydrogen sulphite solution (2×30 ml) and water (30 ml), dried (MgSO₄), evaporated and the residue purified by flash chromatography eluting with 2% ethyl acetate in isohexane to afford the title compound (2.53 g).

LC/MS Rt=3.8 min.

### Ethyl 2′-bromo-6′-methyl-3-biphenylcarboxylate

A mixture of 1-bromo-2-iodo-3-methylbenzene (0.46 g), {3-[(ethyloxy)-carbonyl]phenyl}boronic acid (0.20 g), tetrakis(triphenylphosphine)palladium(0) (60 mg), potassium carbonate (0.71 g), toluene (6 ml) and ethanol (6 ml) was stirred under reflux under nitrogen for 3 h. The mixture was partitioned between water (40 ml) and ether (40 ml) and the aqueous layer extracted with ether (15 ml). The combined ether extracts were dried (MgSO₄), evaporated and the residue purified by flash chromatography eluting with 2% ethyl acetate in isohexane to afford the title compound (0.22 g).

LC/MS Rt=4.1 min, [MH+] 319/321.

### Ethyl 5″-chloro-6′-methyl-2″-[(phenylmethyl)oxy]-1,1′:2′,1″-terphenyl-3-carboxylate

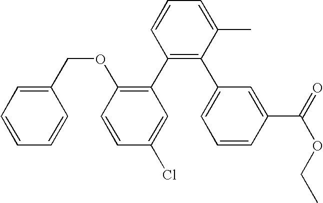

A mixture of ethyl 2′-bromo-6′-methyl-3-biphenylcarboxylate (115 mg), {5-chloro-2-[(phenylmethyl)oxy]phenyl}boronic acid (110 mg), tetrakis-(triphenylphosphine)-palladium(0) (21 mg), potassium carbonate (0.25 g), toluene (2.5 ml) and ethanol (2.5 ml) was stirred under reflux under nitrogen for 4.5 h. The mixture was partitioned between water (15 ml) and ether (15 ml) and the aqueous layer extracted with ether (7 ml). The combined ether extracts were dried (MgSO₄), evaporated and the residue purified by flash chromatography eluting with 2% ethyl acetate in isohexane to afford the title compound (52 mg). LC/MS Rt=4.5 min.

### Example 49

### Sodium 5″-chloro-6′-methyl-2″-[(phenylmethyl)oxy]-1,1′:2′,1″-terphenyl-3-carboxylate

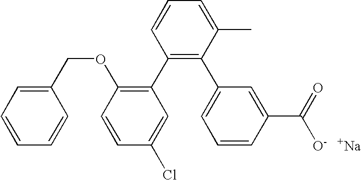

A solution of ethyl 5″-chloro-6′-methyl-2″-[(phenylmethyl)oxy]-1,1′:2′,1″-terphenyl-3-carboxylate (83 mg) in 2M sodium hydroxide solution (0.4 ml) and ethanol (2 ml) was stirred at reflux for 1 h. The mixture was concentrated under reduced pressure and the residue partitioned between water (4 ml) and ether (4 ml). The organic layer was washed with brine (2×3 ml), dried (MgSO₄), evaporated and the residue washed with isohexane and water and dried in vacuo at 50° to afford the title compound (71 mg).

LC/MS Rt=4.2 min, [MH⁺] 427/429.

### Ethyl 6-(5′-Bromo-2′-hydroxy-2-biphenylyl)-2-pyridinecarboxylate

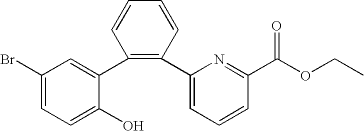

The title compound was prepared in a similar manner to methyl 6-(5′-Bromo-2′-hydroxy-2-biphenylyl)-2-pyridinecarboxylate. Rt=3.27 min [MH⁺]=398, 400.

### [1-(Trifluoromethyl)cyclopropyl]methyl 4-methylbenzenesulfonate

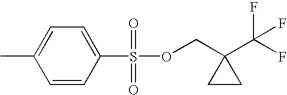

1M Lithium aluminium hydride in tetrahydrofuran (4.55 ml, 4.55 mmol) was slowly added to a stirred solution of 1-(trifluoromethyl)cyclopropanecarboxylic acid (1 g, 6.5 mmol) in diethyl ether(6 ml) at −15° C. The mixture was stirred for 30 minutes, warmed to room temperature, quenched with water and extracted with diethyl ether (×3). The combined organic phases were dried (MgSO₄) and concentrated to ˜5 ml.

The residue was dissolved in dichloromethane (4 ml), pyridine (0.48 ml, 6 mmol) and p-toluenesulfonyl chloride (1.1 g, 6 mmol) were added. The mixture was stirred for 3 hours at room temperature, warmed at 50° C. for 2 hours, cooled to room temperature, then evaporated. Water was added then 2M hydrochloric acid and the mixture was extracted with diethyl ether(×3). The combined organic extracts were dried (MgSO₄) and evaporated to dryness to give 100 mg of the title compound.

¹HNMR CDCl₃ δ: 0.82-0.86(2H, m), 1.10-1.13(2H, m), 2.46(3H, s), 4.09(2H, s), 7.35(2H, d), 7.78(2H, d).

### Ethyl 6-[5′-chloro-2′-({[1-(trifluoromethyl)cyclopropyl]methyl}oxy)-2-biphenylyl]-2-pyridinecarboxylate

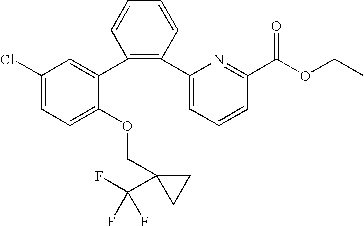

[1-(Trifluoromethyl)cyclopropyl]methyl 4-methylbenzenesulfonate(100 mg, 0.34 mmol), ethyl 6-(5′-chloro-2′-hydroxy-2-biphenylyl)-2-pyridinecarboxylate(100 mg, 0.28 mmol) and potassium carbonate(98 mg, 0.71 mmol) in N,N-dimethylformamide(3 ml), were heated at 90° C. for 3 hours. After cooling the reaction mixture was purified by flash chromatography using 10% of ethyl acetate in hexane. Rt=3.74 min [MH⁺]=476.

The following compounds were prepared from appropriate intermediates in a similar manner to methyl 6-{5′-bromo-2′-[(2-methylbutyl)oxy]-2-biphenylyl}-2-pyridinecarboxylate:

| | Compound name | LC/MS |
|---|---|---|
| 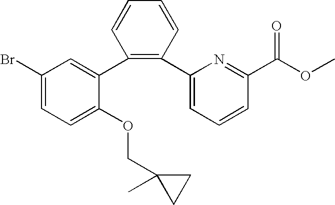 | Methyl 6-(5′-bromo-2′-{[(1- methylcyclopropyl)methyl]oxy}- 2-biphenylyl)-2- pyridinecarboxylate | [MH⁺] =454 Rt = 4.11 min |
| 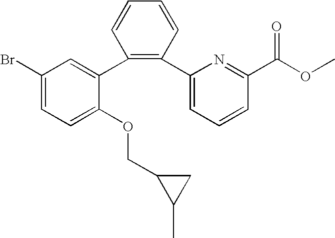 | Methyl 6-(5′-bromo-2′-{[(2- methylcyclopropyl)methyl]oxy}- 2-biphenylyl)-2- pyridinecarboxylate TS103932-186C | [MH⁺] =454 Rt = 4.06 min |
| 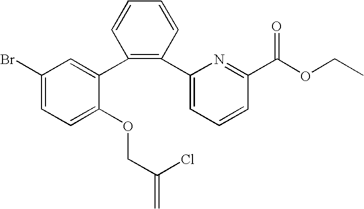 | Ethyl 6-{5′-bromo-2′-[(2- chloro-2-propen-1-yl)oxy]-2- biphenylyl}-2- pyridinecarboxylate | [MH⁺] =474, 476 Rt = 3.71 min |
| 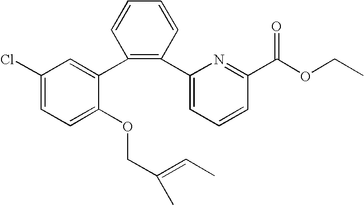 | Ethyl 6-(5′-chloro-2′-{[(2E)-2- methyl-2-buten-1-yl]oxy}-2- biphenylyl)-2- pyridinecarboxylate | [MH⁻] =420, 422 Rt = 3.85 min |
| 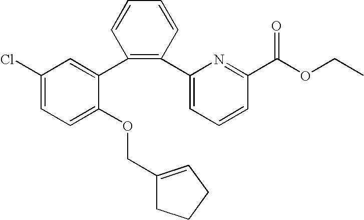 | Ethyl 6-{5′-chloro-2′-[(1- cyclopenten-1-ylmethyl)oxy]- 2-biphenylyl}-2- pyridinecarboxylate | [MH⁺] =434, 436 Rt = 3.84 min |
| 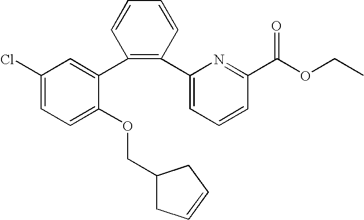 | Ethyl 6-{5′-chloro-2′-[(3- cyclopenten-1-ylmethyl)oxy]- 2-biphenylyl}-2- pyridinecarboxylate | [MH⁺] =434, 436 Rt = 3.91 min |

### Methyl 6-(5′-bromo-2′-{[(2S)-2-methylbutyl]oxy}-2-biphenylyl)-2-pyridinecarboxylate

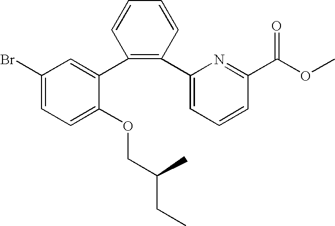

A mixture of methyl 6-(5′-bromo-2′-hydroxy-2-biphenylyl)-2-pyridinecarboxylate (109 mg, 0.286 mmol), (2S)-2-methyl-1-butanol (31 μl, 0.26 mmol), triphenylphosphine (75 mg, 0.286 mmol) and diisopropyl azodicarboxylate (57 μl, 0.286 mmol) in tetrahydrofuran (4 ml) was stirred at room temperature overnight. Triphenylphosphine(75 mg), diisopropyl azodicarboxylate(57 μl)and (2S)-2-methyl-1-butanol (31 μl) were added and the reaction was stirred for another two hours. The mixture was then evaporated and the residue was purified by flash chromatography eluting with ˜5-10% ethyl acetate in hexane to yield the title compound (100 mg). LC/MS: Rt=3.9 min. [MH⁺]=456.

The following compounds were prepared from appropriate intermediates in a similar manner to methyl 6-{5′-bromo-2′-[(2-methyl-2-propen-1-yl)oxy]-2-biphenylyl}-2-pyridinecarboxylate:

| | Compound name | DATA |
|---|---|---|
| 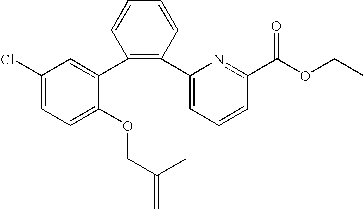 | Ethyl 6-{5′-chloro-2′-[(2- methyl-2-propen-1-yl)oxy]-2- biphenylyl}-2- pyridinecarboxylate | ¹HNMR(CDCl₃)δ: 1.39(3H, t), 1.52(3H, s), 3.99(2H, br s), 4.41- 4.49(2H, m), 4.64(1H, s), 4.75(1H, s), 6.62(1H, d), 7.13(1H, d), 7.22-7.25 (2H, |
| # m), 7.32- 7.38(1H, m), 7.44- 7.47(2H, m), 7.57(1H, t), 7.76-7.77(1H, m), 7.92(1H, d) | | |
| 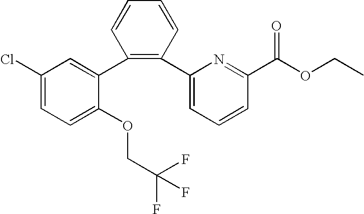 | Ethyl 6-{5′-chloro-2′-[(2,2,2- trifluoroethyl)oxy]-2- biphenylyl}-2- pyridinecarboxylate | ¹HNMR(CDCl₃)δ: 1.36(3H, t), 3.8-4.18(2H, br signal), 4.28-4.40(2H, m), 6.72(1H, d), 7.16- 7.21(2H, m), 7.38-7.40 (2H, m), 7.47- 7.50(2H, m), 7.67- 7.72(2H, m), 7.93(1H, d) |
| 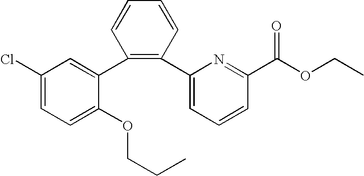 | Ethyl 6-[5′-chloro-2′- (propyloxy)-2-biphenylyl]-2- pyridinecarboxylate | ¹HNMR(CDCl₃)δ: 0.73(3H, t), 1.35- 1.49(5H, m), 3.20- 3.78(2H, br signal), 4.42(2H, q), 6.61(1H, d), 7.14-7.57(7H, m), 7.7- 7.85(1H, m), 7.91(1H, d) |
| 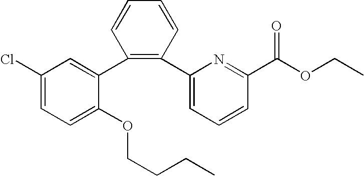 | Ethyl 6-[2′-(butyloxy)-5′- chloro-2-biphenylyl]-2- pyridinecarboxylate | ¹HNMR(CDCl₃)δ: 0.78(3H, t), 1.11- 1.19(2H, m), 1.32- 1.42(5H, m), 3.20- 3.80(2H, br signal), 4.39-4.48(2H, m), 6.61(1H, d), 7.14- 7.47(6H, m), 7.56(1H, t), 7.79(1H, d), 7.91(1H, d) |
| 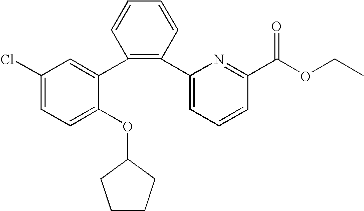 | Ethyl 6-[5′-chloro-2′- (cyclopentyloxy)-2- biphenylyl]-2- pyridinecarboxylate | ¹HNMR(CDCl₃)δ: 1.38- 1.41(8H, m), 1.55- 1.67(3H, m), 4.30- 4.35(1H, m), 4.40- 4.45(2H, m), 6.59(1H, d), 7.13-7.46(6H, m), 7.56(1H, t), 7.79(1H, d), 7.91(1H, d) |
| 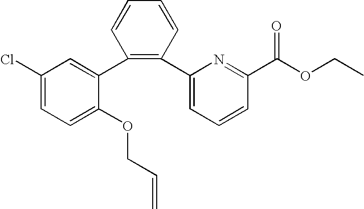 | Ethyl 6-[5′-chloro-2′-(2- propen-1-yloxy)-2- biphenylyl]-2- pyridinecarboxylate | ¹HNMR(CDCl₃)δ: 1.38(3H, t), 3.85- 4.25(2H, br signal), 4.30- 4.50(2H, br signal), 4.96- 5.06(2H, m), 5.55- 5.62(1H, m), 6.59(1H, d), 7.15(1H, d), |
| # 7.23- 7.48(5H, m), 7.58(1H, t), 7.79(1H, d), 7.91(1H, d) | | |
| 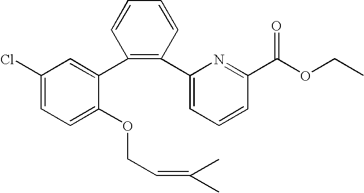 | Ethyl 6-{5′-chloro-2′-[(3- methyl-2-buten-1-yl)oxy]-2- biphenylyl}-2- pyridinecarboxylate | ¹HNMR(CDCl₃)δ: 1.39(3H, t), 1.51(3H, s), 1.63(3H, s), 3.90- 4.25(2H, br signal), 4.30- 4.50(2H, br signal), 4.82- 4.86(1H, m), 6.58(1H, d), 7.15(1H, d), |
| #7.23- 7.48(5H, m), 7.57(1H, t), 7.75-7.82(1H, m), 7.92(1H, d) | | |

### Ethyl 6-{5′-chloro-2′-[(2-chloro-2-propen-1-yl)oxy]-2-biphenylyl}-2-pyridinecarboxylate

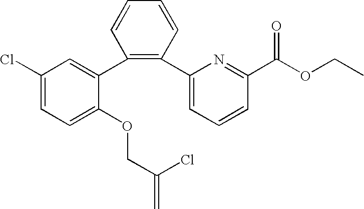

A mixture of ethyl 6-(5′-chloro-2′-hydroxy-2-biphenylyl)-2-pyridinecarboxylate (100 mg, 0.286 mmol), 2-chloro-2-propen-1-ol (21 μl, 0.26 mmol), triphenylphosphine (75 mg, 0.286 mmol) and diisopropyl azodicarboxylate (57 μl, 0.286 mmol) in tetrahydrofuran (4 ml) was stirred at room temperature overnight. The mixture was then warmed at 50° C. for 40 minutes, cooled to room temperature and evaporated. The residue was purified by flash chromatography using ˜10% ethyl acetate in hexane as eluent to yield the title compound (30 mg).

LC/MS: Rt=3.33 min. [MH⁺]=430, 432

The following compounds were prepared from appropriate intermediates in a similar manner to ethyl 6-{5′-chloro-2′-[(2-chloro-2-propen-1-yl)oxy]-2-biphenylyl}-2-pyridinecarboxylate:

| | Compound name | LC/MS |
|---|---|---|
| 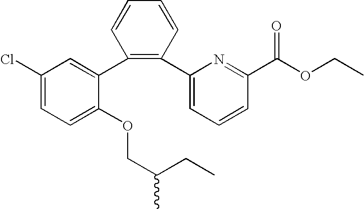 | Ethyl 6-{5′-chloro-2′-[(2- methylbutyl)oxy]-2- biphenylyl}-2- pyridinecarboxylate | [MH⁺] =426, 427 Rt = 3.61 min |
| 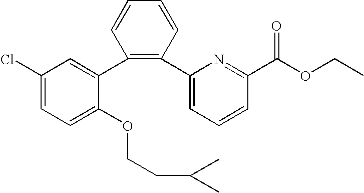 | Ethyl 6-{5′-chloro-2′-[(3- methylbutyl)oxy]-2- biphenylyl}-2- pyridinecarboxylate | [MH⁺] =424, 427 Rt = 3.58 min |
| 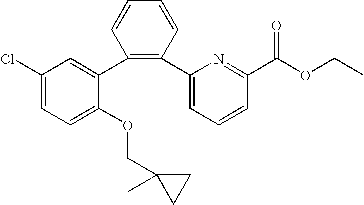 | Ethyl 6-(5′-chloro-2′-{[(1- methylcyclopropyl)methyl]oxy}- 2-biphenylyl)-2- pyridinecarboxylate | [MH⁺] =422, 425 Rt = 3.49 min |

### Ethyl 6-{5′-chloro-2′-[(2-ethylbutyl)oxy]-2-biphenylyl}-2-pyridinecarboxylate

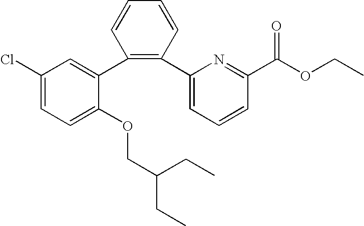

A mixture of ethyl 6-(5′-chloro-2′-hydroxy-2-biphenylyl)-2-pyridinecarboxylate (110 mg, 0.311 mmol), potassium carbonate (107 mg, 0.78 mmol) and 3-(bromomethyl)pentane (48 μl, 0.34 mmol) was stirred in N,N′-dimethylformamide (3 ml) at 90° C. After 3 hours 3-(bromomethyl)pentane (15 μl) was added and the mixture was heated for a further hour. After cooling the reaction mixture was evaporated, diluted with water and extracted with diethyl ether (×3). The combined organic layers were dried (MgSO₄) and evaporated to dryness, the residue was purified by flash chromatography using 5% ethyl acetate in hexane as eluent to yield the title compound (100 mg).

The following compounds were prepared from appropriate intermediates in a similar manner to ethyl 6-{5′-chloro-2′-[(2-ethylbutyl)oxy]-2-biphenylyl}-2-pyridinecarboxylate:

| | |
|---|---|
| 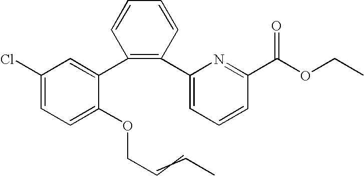 | ethyl 6-{2′-[(2E/Z)-2-buten- 1-yloxy]-5′-chloro-2- biphenylyl}-2- pyridinecarboxylate |

### Example 50

### 6-(5′-Bromo-2′-{[(2S)-2-methylbutyl]oxy}-2-biphenylyl)-2-pyridinecarboxylic acid

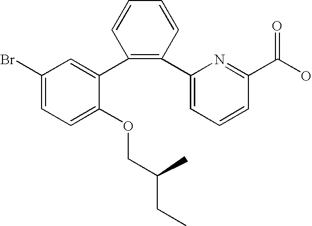

Methyl 6-(5′-bromo-2′-{[(2S)-2-methylbutyl]oxy}-2-biphenylyl)-2-pyridinecarboxylate (100 mg, 0.22 mmol), was dissolved in methanol (3 ml), 2M sodium hydroxide (1 ml) added and the reaction stirred at room temperature for 2 hours. The mixture was evaporated to dryness, diluted with water, acidified with acetic acid and extracted with ethyl acetate (×3). The combined organic layers were dried (MgSO₄) and evaporated to give the title compound (78 mg). LC/MS: Rt=3.74 [MH⁻]=442.
General Hydrolysis Procedure

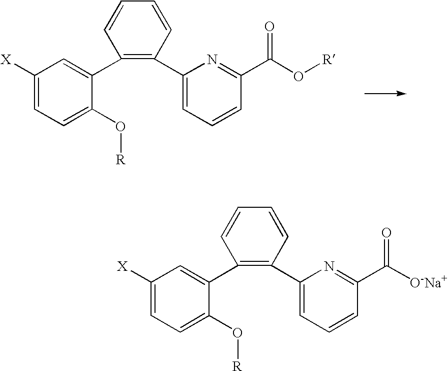

The ester (0.5 mmol) was dissolved in ethanol or methanol (3 ml) and 2M sodium hydroxide (1-2 ml) added. The mixture was stirred from room temperature to reflux for from 30 minutes to 20 hours until the reaction was complete by tlc. The mixture was diluted with water and extracted with ethyl acetate (×3). The combined organic layers were dried and evaporated to dryness to give the title compound.

The following compounds were prepared by the general hydrolysis procedure:

| EXAMPLE | | COMPOUND NAME | LC/MS |
|---|---|---|---|
| 51 | 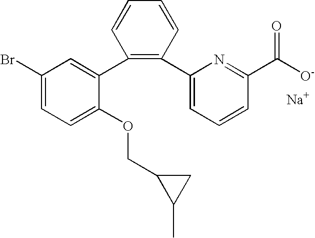 | Sodium 6-(5′-bromo- 2′-{[(2- methylcyclopropyl) methyl]oxy}-2- biphenylyl)-2- pyridinecarboxylate | [MH⁻] = 439 Rt = 3.24 min |
| 52 | 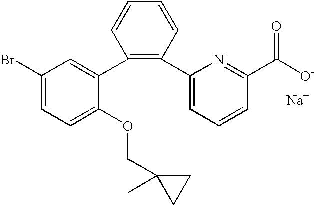 | Sodium 6-(5′-bromo- 2′-{[(1- methylcyclopropyl) methyl]oxy}-2- biphenylyl)-2- pyridinecarboxylate | [MH⁻] = 439 Rt = 3.27 min |
| 53 | 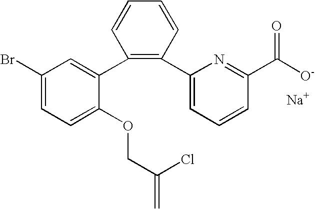 | Sodium 6-{5′-bromo- 2′-[(2-chloro-2- propen-1-yl)oxy]-2- biphenylyl}-2- pyridinecarboxylate | [MH⁺] =446, 448 Rt = 3.48 min |
| 54 | 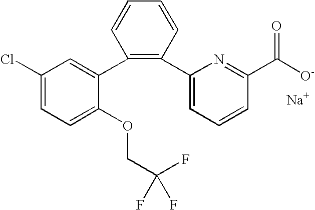 | Sodium 5″-chloro-2″- [(2,2,2- trifluoroethyl)oxy]- 1,1′:2′,1″-terphenyl-3- carboxylate | [MH⁻] =406, 408 Rt = 2.95 min |
| 55 | 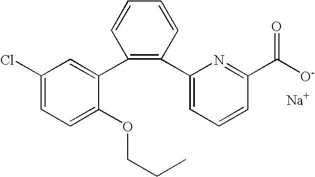 | Sodium 6-[5′-chloro- 2′-(propyloxy)-2- biphenylyl]-2- pyridinecarboxylate | [MH⁻] =366, 368 Rt = 3.08 min |
| 56 | 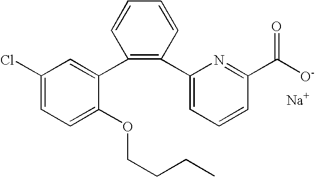 | Sodium 6-[2′- (butyloxy)-5′-chloro-2- biphenylyl]-2- pyridinecarboxylate | [MH⁻] =380, 382 Rt = 3.22 min |
| 57 | 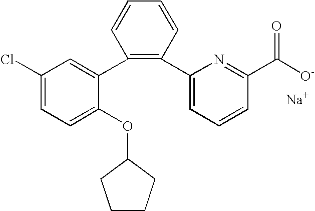 | Sodium 6-[5′-chloro- 2′-(cyclopentyloxy)-2- biphenylyl]-2- pyridinecarboxylate | [MH⁻] =392, 394 Rt = 3.22 min |
| 58 | 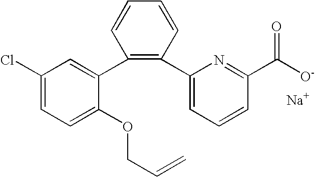 | Sodium 6-[5′-chloro- 2′-(2-propen-1-yloxy)- 2-biphenylyl]-2- pyridinecarboxylate | [MH⁻] =364, 366 Rt = 2.96 min |
| 59 | 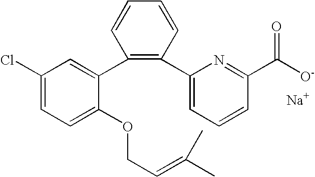 | Sodium 6-{5′-chloro- 2′-[(3-methyl-2-buten- 1-yl)oxy]-2- biphenylyl}-2- pyridinecarboxylate | [MH⁻] =392, 394 Rt = 3.19 min |
| 60 | 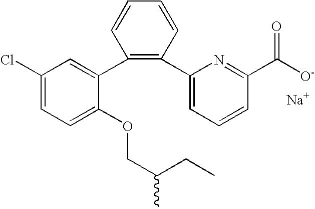 | Sodium 6-{5′-chloro- 2′-[(2- methylbutyl)oxy]-2- biphenylyl}-2- pyridinecarboxylate | [MH⁻] =394, 396 Rt = 3.35 min |
| 61 | 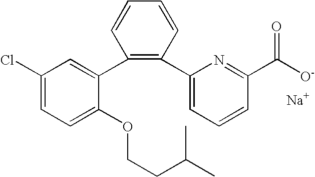 | Sodium 6-{5′-chloro- 2′-[(3- methylbutyl)oxy]-2- biphenylyl}-2- pyridinecarboxylate | [MH⁻] =394, 396 Rt = 3.31 min |
| 62 | 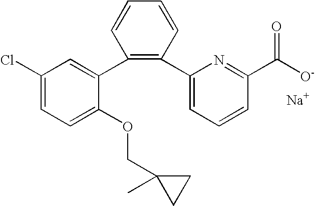 | Sodium 6-(5′-chloro- 2′-{[(1- methylcyclopropyl) methyl]oxy}-2- biphenylyl)-2- pyridinecarboxylate | [MH⁻] =392, 394 Rt = 3.22 min |
| 63 | 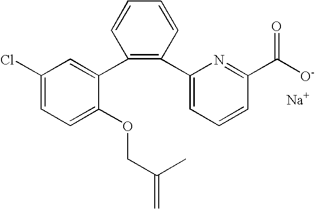 | Sodium 6-{5′-chloro- 2′-[(2-methyl-2- propen-1-yl)oxy]-2- biphenylyl}-2- pyridinecarboxylate | [MH⁻] =378, 380 Rt = 3.46 min |
| 64 | 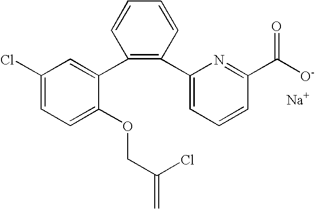 | Sodium 6-{5′-chloro- 2′-[(2-chloro-2- propen-1-yl)oxy]-2- biphenylyl}-2- pyridinecarboxylate | [MH⁻] = 398 Rt = 3.05 min |
| 65 | 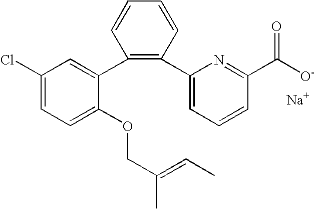 | Sodium 6-(5′-chloro- 2′-{[(2E)-2-methyl-2- buten-1-yl]oxy}-2- biphenylyl)-2- pyridinecarboxylate | [MH⁻] =392, 394 Rt = 3.47 min |
| 66 | 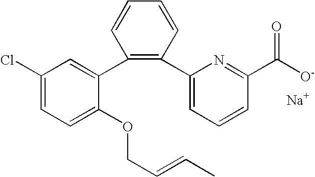 | Sodium 6-{2′-[(2E/Z)- 2-buten-1-yloxy]-5′- chloro-2-biphenylyl}- 2-pyridinecarboxylate | [MH⁻] =378, 380 Rt = 3.34 min |
| 67 | 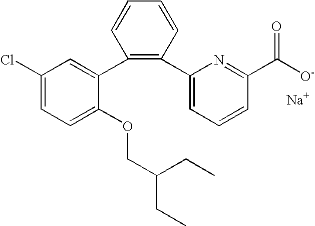 | Sodium 6-{5′-chloro- 2′-[(2-ethylbutyl)oxy]- 2-biphenylyl}-2- pyridinecarboxylate | [MH⁻] =408, 410 Rt = 3.65 min |
| 68 | 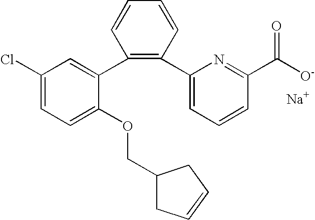 | Sodium 6-{5′-chloro- 2′-[(3-cyclopenten-1- ylmethyl)oxy]-2- biphenylyl}-2- pyridinecarboxylate | [MH⁻] =404, 406 Rt = 3.66 min |
| 69 | 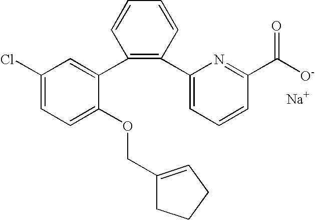 | Sodium 6-{5′-chloro- 2′-[(1-cyclopenten-1- ylmethyl)oxy]-2- biphenylyl}-2- pyridinecarboxylate | [MH⁻] =404, 406 Rt = 3.63 min |
| 70 | 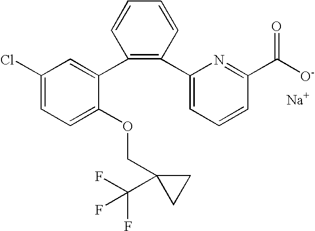 | Sodium 6-[5′-chloro- 2′-({[1-(trifluoromethyl) cyclopropyl]methyl}oxy)-2-biphenylyl]-2- pyridinecarboxylate | [MH⁻] =446, 448 Rt = 3.57 min |

It is to be understood that the present invention covers all combinations of particular and preferred subgroups described herein above.

Assays for Determining Biological Activity

The compounds of formula (I) can be tested using the following assays to demonstrate their prostanoid antagonist or agonist activity in vitro and in vivo and their selectivity. The prostaglandin receptors investigated are DP, EP₁, EP₂, EP₃, EP₄, FP, IP and TP.

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-4, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 100 μM flurbiprofen and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing fluo-4 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (pIC₅₀) may then be estimated.

Binding Assay for the Human Prostanoid EP₁ Receptor

Competition assay using [³H]-PGE2.

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5 mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10 s burst at full setting), centrifuged at 48,000×g for 20 mins and the pellet containing the membrane fraction is washed three times by suspension and centrifugation at 48,000×g for 20 mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at −80° C. until required.

For the binding assay the cell membranes, competing compounds and [³H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques to determine the concentration of compound producing 50% inhibition of specific binding (IC₅₀).

By application of these techniques, compounds of the Examples had an antagonist pIC₅₀ value of 6.0 or greater at EP₁ receptors.

No toxicological effects are indicated/expected when a compound (of the invention) is administered in the above mentioned dosage range.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 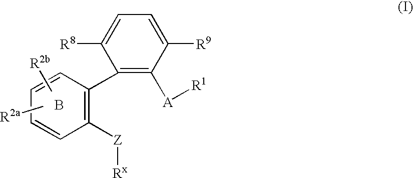
wherein:
A is an optionally substituted aryl, or an optionally substituted 5- or 6-membered heterocyclyl ring, or an optionally substituted bicyclic heterocyclyl group;
B is a phenyl or pyridyl ring;
Z is O, S, SO, or SO₂;
R¹ is CO₂H, CN, CONR⁵R⁶, CH₂CO₂R⁴, SO₂NR⁵R⁶, NR⁴CONR⁵R⁶, COalkyl, 2H-tetrazol-5-yl-methyl, optionally substituted SO₂alkyl optionally substituted bicyclic heterocycle or optionally substituted heterocyclyl;
R^{2a} and R^{2b} are each independently selected from hydrogen, halogen, CONR⁵R⁶, CN, SO₂alkyl, SR⁵, NO₂, optionally substituted alkyl, optionally substituted alkoxy, optionally substituted aryl, and optionally substituted heteroaryl;
R^{x} is optionally substituted alkyl wherein 1 or 2 of the non-terminal carbon atoms are optionally replaced by a group independently selected from NR⁴, O and SOₙ, wherein n is 0, 1 or 2; optionally substituted alkenyl; or optionally substituted alkynyl: or R^{x} is optionally substituted CQ^{a}Q^{b}-heterocyclyl; optionally substituted CQ^{a}Q^{b}-bicyclic heterocyclyl; or optionally substituted CQ^{a}Q^{b}-aryl;
R⁴ is hydrogen or an optionally substituted alkyl;
R⁵ is hydrogen or an optionally substituted alkyl;
R⁶ is hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b} heteroaryl or COR⁷;
R⁷ is optionally substituted alkyl, optionally substituted heteroaryl or optionally substituted aryl;
R⁸ and R⁹ are each independently selected from hydrogen, halogen, CF₃, C₁₋₃alkoxy and C₁₋₃alkyl;
Q^{a} and Q^{b} are each independently selected from hydrogen and CH₃;
and derivatives thereof;
wherein:
when A is a 6-membered ring the R¹ substituent and the above-defined 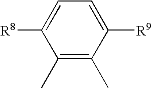
group are attached to carbon atoms 1,2- or 1,3- relative to each other, and when A is a five-membered ring or bicyclic heterocyclyl group the R¹ substituent and the above-defined 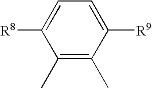
group are attached to substitutable carbon atoms 1,2- or 1,3- relative to each other; and
neither R^{2a} nor R^{2b} are ortho to the Z substituent; and
R^{x} is not 4-(tert-butyl)benzyl or 4-methylpent-1-yl; and
when R¹ is CONH₂, it is ortho to the 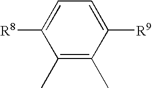
group; and
when R¹ is SO₂NR⁵R⁶, then R⁵ is hydrogen and R⁶ is COR⁷;
provided that the compound of formula (I) is not:
2-benzyloxy-5-chloro-[1,1′;2′,1″]terphenyl-3″-carboxylic acid;
(2-benzyloxy-5-chloro-[1,1′;2′,1″]terphenyl-3″-yl)-acetic acid;
(2-benzyloxy-5-chloro[1,1′;2′,1″]terphenyl-2″-yl)acetic acid;
(2-benzyloxy-5-chloro[1,1′;2′,1″]terphenyl-4″-yl)acetic acid;
5″-acetylamino-2-benzyloxy-5-chloro[1,1′;2′,1″]terphenyl-3″-carboxylic acid;
2-benzyloxy-5-chloro-5″-propionylamino[1,1′;2′,1″]terphenyl-3″-carboxylic acid;
2-benzyloxy-6-chloro-5″-(2-methylpropanoylamino)-[1,1′;2′,1″]terphenyl-3″-carboxylic acid;
2-benzoyloxy-5″-butyrylamino-5-chloro[1,1′;2′1″]terphenyl-3″-carboxylic acid;
2-benzyloxy-5-chloro-5″-[(1-phenyl-methanoyl)amino]-[1,1′;2′,1″]terphenyl-3″-carboxylic acid;
2-benzyloxy-5-chloro-5″-methanesulfonylamino-[1,1′;2′,1″]terphenyl-3″-carboxylic acid
5″-amino-2-benzyloxy-5-chloro[1,1′;2′,2″]-3″-carboxylic acid;
2-benzyloxy-5″-butyrylamino-5-trifluoromethyl[1,1′;2′,1″]terphenyl-3″-carboxylic acid-3-carboxylic acid;
2-benzyloxy-4″-chloro[1,1′;2′,1″]terphenyl 2″-carboxylic acid;
2-benzyloxy-5″-fluoro-[1,1′;2′,1″]terphenyl-2″-carboxylic acid;
2-benzyloxy-4″-fluoro-[1,1′;2′,1″]terphenyl-2″-carboxylic acid;
2″-benzyloxy-5-fluoro-[1,1′;2′,1″]terphenyl-3-carboxylic acid;
4″-amino-2-benzyloxy-[1,1′;2′,1″]terphenyl-3″-carboxylic acid;
5″-acetylamino-2-benzyloxy-[1,1′;2′,1″]terphenyl-2″-carboxylic acid;
2-benzyloxy-5-chloro-[1,1′;2′,1″]terphenyl-2″-carboxylic acid;
2-benzyloxy-[1,1′;2′,1″]terphenyl-3-carboxylic acid;
2-benzyloxy-5-chloro-[1,1′;2′,1″]terphenyl-2″-carboxylic acid amide;
5-(2-benzyloxy-5-chloro-[1,1′;2′1″]terphenyl-3″-yl)-1H-tetrazole;
N-[1-(2-benzyloxy-5-chloro[1,1′;2′,1″]terphenyl-2″-yl)-methanoyl]-benzenesulfonamide;
2-benzyloxy-[1,1′;2′,1″]terphenyl-4′-sulfonic acid (1-phenyl-methanoyl)-amide;
2-benzyloxy-[1,1′;2′,1″]terphenyl-4″-sulfonic acid [1-(4-nitro-phenyl)-methanoyl]-amide;
2-benzyloxy-[1,1′;2′,1″]terphenyl-3″-sulfonic acid acetyl-amide;
5-chloro-2-(3-methyl-butoxy)-[1,1′;2′,1″]terphenyl-3″-carboxylic acid;
5-chloro-2-(4-fluoro-benzyloxy)-[1,1′;2′,1″]terphenyl-3″-carboxylic acid;
5-chloro-2-(2,4-difluoro-benzyloxy)-[1,1′;2′,1″]terphenyl-3′-carboxylic acid;
5-chloro-2-(4-chloro-benzyloxy)-[1,1′;2′,1″]terphenyl-3″carboxylic acid;
5-chloro-2-(2-fluoro-4-chloro-benzyloxy)-[1,1′;2′,1″]terphenyl-3″-carboxylic;
5-chloro-2-(4-isobutoxy)-[1,1′,2′,1″]terphenyl-3″-carboxylic acid;
5-chloro-2-(pyridin-2-ylmethoxy)-[1,1′;2′,1″]terphenyl-3″-carboxylic acid;
5-chloro-2-(pyridin-4-ylmethoxy)-[1,1′;2′,1″]terphenyl-3″-carboxylic acid;
5-chloro-2-(pyridin-3-ylmethoxy)-[1,1′;2′,1″]terphenyl-3″-carboxylic acid;
5-chloro-2-cyclohexylmethoxy-[1,1′;2′,1″]terphenyl-3′-′carboxylic acid;
5-chloro-2-(thiophen-3-ylmethoxy)-[1,1′;2′,1″]terphenyl-3″-carboxylic acid;
5-chloro-2-(thiophen-2-ylmethoxy)-[1,1′;2′,1″]terphenyl-3″-carboxylic acid;
5-chloro-2-cyclopentylmethoxy-[1,1′;2′,1″]terphenyl-3″-carboxylic acid;
5-chloro-2-propoxy-[1,1′;2′,1″]terphenyl-3″-carboxylic acid;
2-butoxy-5-chloro-[1,1′;2′,1″]terphenyl-3″-carboxylic acid;
5-chloro-2-isopropoxy-[1,1′;2′,1″]terphenyl-3″-carboxylic acid;
5-chloro-2-isobutoxy-[1,1′;2′,1″]terphenyl-2″-carboxylic acid;
2″-{[(4-fluorophenyl)methyl]oxy}-5-[(Methyloxy)carbonyl]-5″-(trifluoromethyl)-1,1′:2′,1″-terphenyl-3-carboxylic acid;
5-chloro-2″-[(phenylmethyl)oxy]-1,1′:2′,1″-terphenyl-2-carboxylic acid;
4-(methoxy)-2″-[(phenylmethyl)oxy]-1′1′:2′,1″-terphenyl-2-carboxylic acid;
2″-{[(2,4-difluorophenyl)methyl]oxy}-4-(propanoylamino)-5″-(trifluoromethyl)-1,1′:2′,1″-terphenyl-2-carboxylic acid;
2″-{[(2,4-difluorophenyl)methyl]oxy}-4-[(2-methylpropanoyl)amino]-5″-(trifluoromethyl)-1,1′:2′,1″-terphenyl-2-carboxylic acid;
5-(2-oxo-1-pyrrolidinyl)-2″-[(phenylmethyl)oxy]-5″-(trifluoromethyl)-1,1′:2′,1″-terphenyl-3-carboxylic acid;
2″-{[(4-fluorophenyl)methyl]oxy}-5″-(trifluoromethyl)-1,1′:2′,1″-terphenyl-3,5-dicarboxylic acid;
2″-{[(4-fluorophenyl)methyl]oxy}-5-{[(2-methylpropyl)amino]carbonyl}-5″-(trifluoromethyl)-1,1′:2′1″-terphenyl-3-carboxylic acid;
6-[2′-{[(4-fluorophenyl)methyl]oxy}-5′-(trifluoromethyl)-2-biphenylyl]-2-pyrazinecarboxylic acid;
2″-{[(4-fluorophenyl)methyl]oxy}-5-(propanoylamino)-5″-(trifluoromethyl)-1,1′:2′,1″-terphenyl-3-carboxylic acid;
2″-[(phenylmethyl)oxy]-5-(propanoylamino)-5″-(trifluoromethyl)-1,1′:2′,1″-terphenyl-3-carboxylic acid;
2″-{[(2,4-difluorophenyl)methyl]oxy}-5-(propanoylamino)-5″-(trifluoromethyl)-1,1′:2′,1″-terphenyl-3-carboxylic acid;
5″-chloro-5-{[(methyloxy)acetyl]amino}-2″-[(phenylmethyl)oxy]-1,1′:2′,1″-terphenyl-3-carboxylic acid;
5″-chloro-2″-[(phenylmethyl)oxy]-5-[(2-thienylacetyl)amino]-1,1′:2′,1″-terphenyl-3-carboxylic acid;
5″-chloro-2″-[(phenylmethyl)oxyl-5-({[(phenylmethyl)oxy]acetyl}amino)-1,1′:2′,1″-terphenyl-3-carboxylic acid;
5-{[(1-acetyl-4-piperidinyl)carbonyl]amino}-5″-chloro-2″-[(phenylmethyl)oxy]-1,1′:2′,1″-terphenyl-3-carboxylic acid;
5″-chloro-5-[(phenylacetyl)amino]-2″-[(phenylmethyl)oxy]-1,1′:2′,1″-terphenyl-3-carboxylic acid;
5″-chloro-5-{[(3,5-dimethyl-4-isoxazolyl)carbonyl]amino}-2″-[(phenylmethyl)oxy]-1,1′:2′,1″-terphenyl-3-carboxylic acid;
5″-chloro-5-[(3-methylbutanoyl)amino]-2″-[(phenylmethyl)oxy]-1,1′:2′,1″-terphenyl-3-carboxylic acid;
5″-chloro-5-(glycylamino)-2″-[(phenylmethyl)oxy]-1,1′:2′,1″-terphenyl-3-carboxylic acid;
2″-[(penylmethyl)oxy]-4-(propanoylamino)-1,1′:2′,1″-terphenyl-2-carboxylic acid;
4-[(2-methylpropanoyl)amino]-2″-[(phenylmethyl)oxy]-1,1′:2′,1″-terphenyl-2-carboxylic acid;
5-cyano-2″-{[(2,4-difluorophenyl)methyl]oxy}-5″-(trifluoromethyl)-1,1′:2′,1″-terphenyl-3-carboxylic acid;
5″-bromo-5-cyano-2″-[(phenylmethyl)oxy]-1,1′:2′,1″-terphenyl-3-carboxylic acid;
5-cyano-2″-[(phenylmethyl)oxy]-5″-(trifluoromethyl)-1,1′:2′,1″-terphenyl-3-carboxylic acid;
5-(aminocarbonyl)-2″-{[(4-fluorophenyl)methyl]oxy}-5″-(trifluoromethyl)-1,1′:2′,1″-terphenyl-3-carboxylic acid;
2″-{[(4-fluorophenyl)methyl]oxy}-5-{[(2-hydroxyethyl)amino]carbonyl}-5″-(trifluoromethyl)-1,1′:2′,1″-terphenyl-3-carboxylic acid;
2″-{[(4-fluorophenyl)methyl]oxy}-5-{[(3-pyridinylmethyl)amino]carbonyl}-5″-(trifluoromethyl)-1,1′:2′,1″-terphenyl-3-carboxylic acid;
6-{5′-chloro-2′-[(phenylmethyl)oxy]-2-biphenylyl}-2-pyridinecarboxylic acid;
6-(5′-chloro-2′-{[(4-fluorophenyl)methyl]oxy}-2-biphenylyl)-2-pyridinecarboxylic acid;
6-(5′-chloro-2′-{[(2,4-difluorophenyl)methyl]oxy}-2-biphenylyl)-2-pynidinecarboxylic acid;
2-[2′-{[(4-fluorophenyl)methyl]oxy}-5′-(trifluoromethyl)-2-biphenylyl]-4-pyridinecarboxylic acid;
3-amino-6-[2′-{[(4-fluorophenyl)methyl]oxy}-5′-(trifluoromethyl)-2-biphenylyl]-2-pyrazinecarboxylic acid;
4-(acetylamino)-2″-[(phenylmethyl)oxy]-5″-(trifluoromethyl)-1,1′:2′,1″-terphenyl-2-carboxylic acid;
4-(acetylamino)-2″-{[(4-fluorophenyl)methyl]oxy}-5″-(trifluoromethyl)-1,1′:2′,1″-terphenyl-2-carboxylic acid;
4-(acetylamino)-2″-{[(2,4-difluorophenyl)methyl]oxy}-5″-(trifluoromethyl)-1,1′:2′,1″-terphenyl-2-carboxylic acid;
4-methyl-2″-[(phenylmethyl)oxy]-1,1′:2′,1″-terphenyl-2-carboxylic acid;
2″-{[(2,4-difluorophenyl)methyl]oxy}-4-methyl-5″-(trifluoromethyl-1,1′:2′,1″-terphenyl-2-carboxylic acid;
2-benzyloxy-5-fluoro[1,1′,2,2′]terphenyl-3″-carboxylic acid;
2-(4-fluorobenzyl)oxy-5-fluoro[1,1′,2,2′]terphenyl-3″-carboxylic acid;
2-(2,4-difluorobenzyl)oxy-5-fluoro[1,1′,2,2′]terphenyl-3″-carboxylic acid;
2′-{5-chloro-2-(benzyloxy)-3-pyridinyl]-3-biphenylcarboxylic acid;
2′-{6-chloro-3-[(phenylmethyl)oxy]-2-pyridinyl}-3-biphenylcarboxylic acid;
5-amino-2′-{6-chloro-3-[(phenylmethyl)oxy]-2-pyridinyl}-3-biphenylcarboxylic acid;
5-{2′-[(phenylmethyl)oxy]-2-biphenylyl}-3-pyridinecarboxylic acid;
4″-chloro-2″-[(phenylmethyl)oxy]-1,1′:2′,1″-terphenyl-2-carboxylic acid; or
6″-fluoro-2-benzyloxy-[1,1′;2′,1″]terphenyl-3″-carboxylic acid.

2. A compound according to claim 1 which is a compound of formula (Ia): 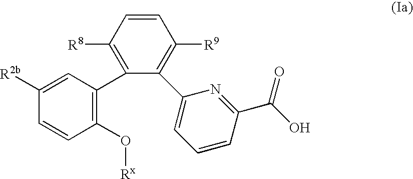
wherein:
R^{2b} is selected from CF₃, chloro and bromo;
R^{x} is optionally substituted alkyl wherein 1 or 2 of the non-terminal carbon atoms are optionally replaced by a group independently selected from NR⁴, O and SOₙ, wherein n is 0, 1 or 2; optionally substituted alkenyl; or optionally substituted alkynyl: or R^{x} is optionally substituted CQ^{a}Q^{b}-heterocyclyl; optionally substituted CQ^{a}Q^{b}-bicyclic heterocyclyl; or optionally substituted CQ^{a}Q^{b}-aryl;
R⁴ is hydrogen or C₁₋₆alkyl;
R⁸ and R⁹ are independently selected from hydrogen, halogen, CF₃, C₁₋₃alkoxy and C₁₋₃alkyl;
Q^{a} and Q^{b} are independently selected from hydrogen and CH₃;
or a derivative thereof;
with the proviso that:
R^{x} is not 4-(tert-butyl)benzyl or 4-methylpent-1-yl; and
the compound of formula (Ia) is not:
6-{5′-chloro-2′-[(phenylmethyl)oxy]-2-biphenylyl}-2-pyridinecarboxylic acid;
6-(5′-chloro-2′-{[(4-fluorophenyl)methyl]oxy}-2-biphenylyl)-2-pyridinecarboxylic acid; or
6-(5′-chloro-2′-{[(2,4-difluorophenyl)methyl]oxy}-2-biphenylyl)-2-pyridinecarboxylic acid.

3. A compound according to claim 1 which is a compound of formula (Ib): 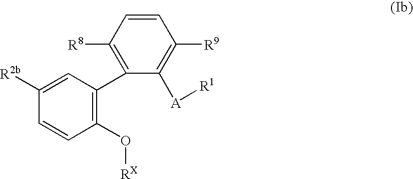
wherein:
A represents an optionally substituted aryl, or an optionally substituted 5- or 6-membered heterocyclyl ring, or an optionally substituted bicyclic heterocyclyl group;
R¹ is CO₂H;
R^{2b} is selected from CF₃, chloro and bromo;
R^{x} represents optionally substituted alkyl wherein 1 or 2 of the non-terminal carbon atoms may also be optionally replaced by a group independently selected from NR⁴, O and SOₙ, wherein n is 0, 1 or 2; or R^{x} represents optionally substituted CQ^{a}Q^{b}-heterocyclyl, optionally substituted CQ^{a}Q^{b}-bicyclic heterocyclyl, optionally substituted CQ^{a}Q^{b} alkenyl, optionally substituted CQ^{a}Q^{b}alkynyl, or optionally substituted CQ^{a}Q^{b}-aryl;
R⁴ is hydrogen or C₁₋₆alkyl;
R⁸ represents hydrogen, halogen, CF₃, C₁₋₃alkoxy or C₁₋₃alkyl;
R⁹ represents halogen, CF₃, C₁₋₃alkoxy or C₁₋₃alkyl; and
Q^{a} and Q^{b} are independently selected from hydrogen and CH₃;
or a derivative thereof;
with the proviso that R^{x} is not 4-(tert-butyl)benzyl or 4-methylpent-1-yl.

4. (canceled)

5. A pharmaceutical composition comprising a compound according to claim 1 or a pharmaceutically acceptable derivative thereof together with a pharmaceutical carrier and/or excipient.

6.-7. (canceled)

8. A method of treating a human or animal subject suffering from a condition which is mediated by the action of PGE₂ at EP₁ receptors which comprises administering to said subject an effective amount of a compound according to claim 1 or a pharmaceutically acceptable derivative thereof.

9. A method of treating a human or animal subject suffering from a pain, or an inflammatory, immunological, bone, neurodegenerative or renal disorder, which method comprises administering to said subject an effective amount of a compound according to claim 1 or a pharmaceutically acceptable derivative thereof.

10. A method of treating a human or animal subject suffering from inflammatory pain, neuropathic pain or visceral pain which method comprises administering to said subject an effective amount of a compound according to claim 1 to or a pharmaceutically acceptable derivative thereof.

11.-13. (canceled)
